(19) United States
(12) Patent Application Publication
(10) Pub. No.: US 2023/0181581 A1
(43) Pub. Date: Jun. 15, 2023

(54) CIRCULATING B CELL SUBPOPULATIONS IN INDOLENT B CELL LYMPHOMA

(71) Applicant: Alexion Pharmaceuticals, Inc., Boston, MA (US)

(72) Inventors: Gregory Coffey, San Carlos, CA (US); Matthew Birrell, Lafayette, CA (US); Pamela B. Conley, Palo Alto, CA (US); Andreas H. Betz, Mountain View, CA (US)

(73) Assignee: Alexion Pharmaceuticals, Inc., Boston, MA (US)

(21) Appl. No.: 17/924,610

(22) PCT Filed: May 20, 2021
(86) PCT No.: PCT/US2021/033482

§ 371 Date: Nov. 10, 2022

Related U.S. Application Data
(60) Provisional application No. 63/030,095, filed on May 26, 2020.

(51) Int. Cl. A61K 31/506; G01N 33/574; A61P 35/00; A61P 7/00

(52) U.S. Cl. CPC A61K 31/506; A61P 7/00; A61P 35/00; G01N 33/57407

(57) ABSTRACT

Methods for treating B cell lymphomas are provided. B cell lymphomas patients suitable for treatments can be identified based on the baseline B cell subset frequencies. For instance, increased frequency of transitional (CD10+) B cells within total nave B cells or within total B cells predicts poor response to kinase inhibitors. By contrast, having an increased nave B cells to total B cells frequency without an increased transitional (CD10+) B cell frequency predicts good response to the kinase inhibitors. Having a decreased frequency of nave B cells of the total B cell population with a corresponding increase in frequency of memory switched and double negative B cells of the total B cell population also predicts good response to the kinase inhibitors. Once the patients are identified, the patients can be suitably treated with the kinase inhibitors such as cerdulatinib.

### CROSS REFERENCE TO RELATED APPLICATIONS

This application claims the benefit of U.S. Provisional Application No. 63/030,095, filed May 26, 2020, the contents of which is incorporated by reference herein in its entirety.

### FIELD

The present disclosure relates generally to methods of treating B cell lymphomas, including indolent non-Hodgkin lymphomas, based on circulating normal B cell subset frequency distributions.

### BACKGROUND

Some therapeutic agents have been developed to target kinases within the B cell receptor (BCR) mediated signaling to treat B cell lymphomas. But some patients fail standard therapies for chronic lymphocytic leukemia (CLL), small lymphocytic lymphoma (SLL), diffuse large B cell lymphoma (DLBCL) and follicular lymphoma (FL), and have limited treatment options. There are needs to assess the impacts of these therapeutic agents on B cell subpopulations, as well as the effectiveness of the therapeutic agents on patients with different B cell subpopulations.

### SUMMARY

The present disclosure relates to the use of a kinase inhibitor in the treatment of B cell lymphomas. Also provided are methods for identifying patients suitable for such treatments, and for monitoring the outcome of the treatment.

In some embodiments, provided herein is a method for predicting the response to a treatment with a B cell lymphoma based on baseline (pre-treatment) frequency distributions of circulating peripheral blood B cell subsets. The treatment may be a kinase inhibitor, such as a PI3K inhibitor, a SYK inhibitor, a JAK inhibitor, and a BTK inhibitor. A patient predicated to have positive response to the treatment may be one suffering from the B cell lymphoma, and having a B cell subset frequency distribution similar to those in Group 2 or Group 3 identified in Example 1. By contrast, a B cell lymphoma patient having a B cell subset distribution similar to those in Group 1 may respond poorly to the treatment.

As shown in the experimental examples, both Group 1 and Group 2 patients, at baseline, have increased (or conveniently referred to as “high”) naïve B cell frequencies within total B cells, as compared to healthy individuals. Unlike Group 1 patients who also have increased transitional (CD10+) B cell frequencies within the total B cell population and naive B cell population, however, Group 2 patients have similar (or conveniently referred to as “normal” or “unchanged”) transitional (CD10+) B cell frequencies, as compared to healthy individuals.

Patients in Group 3 have a more distinct B cell subset distribution pattern. They have decreased (or conveniently referred to as “low”) naïve B cell frequencies within total B cells, as compared to healthy individuals. Also important, patients of Group 3 have increased (or “high”) memory switched and/or double negative B cell frequencies within the total B cell populations.

Therefore, by comparing the B cell subset distribution of a candidate B cell lymphoma patient to those of Groups 1-3, one can predict the outcome of the candidate patient if being treated with the kinase inhibitor. It should be noted that the kinase inhibitor can be a dual inhibitor, meaning the inhibitor inhibits one or more of PI3K, SYK, JAK, or BTK. An example dual inhibitor is cerdulatinib.

Cerdulatinib is a small molecule, ATP-competitive, reversible inhibitor of both SYK and JAK family members and is described in U.S. Pat. 8,138,339 and U.S. Pat. 8,501,944. Cerdulatinib has a chemical name of 4-(cyclopropylamino)-2-((4-(4-(ethylsulfonyl)piperazin-1-yl)phenyl)amino)pyrimidine-5-carboxamide, and the structure of formula I:

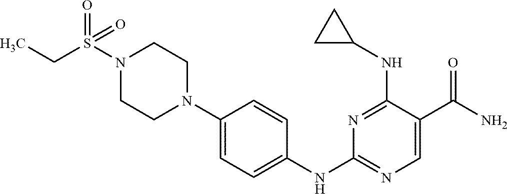 [SMILES: CCS(=O)(=O)N1CCN(c2ccc(Nc3ncc(C(N)=O)c(NC4CC4)n3)cc2)CC1]

### BRIEF DESCRIPTION OF THE DRAWINGS

FIG. 1 includes plots showing flow cytometry gating strategy for identification of B cell subpopulations. Shown is the general gating flow for the identification of B cell subsets from PBMC’s of a healthy volunteer. The top row depicts the forward scatter area (FSC-A) and side scatter (SSC) of the PBMC population, with a gate to select out cellular debris. Next is a FSC-height (FSC-H) b FSC-A gating on single cells, followed by aqua viability stain by CD19 to gate on live B cells. CD20 by HLA-DR confirms that the CD19 gate represents B cells. Below is a CD27 by IgD gate plot, enabling gating on double negative (DN), naïve, switched memory (SM), and non-switched memory (NSM), as defined in the diagram. From the SM gate the CD27 high population is identified, representing late differentiation, and CD38 by CD138 to identify plasma cells, which were rarely seen as expected. IgM by IgG separates out the IgM switched and IgG switched memory B cells. From the naïve B cell gate CD10 positive transitional B cells are identified. The table depicts details of the antibodies used.

FIGS. 2A-2B show plots for principle component analysis (PCA) of baseline circulating B cell subpopulations in iNHL patients identifies three distinct populations with differential response to cerdulatinib. In FIG. 2A, results of the PCA are shown in the top left. The x-axis depicts principle component 1 (PC1, with influence of specific loadings shown in the bar chart, bottom left), and the y-axis depicts principle component 2 (PC2, with influence of specific loadings shown in the bar chart, bottom right). Each symbol represents an individual healthy volunteer or iNHL patient, with best response to therapy indicated, including complete responders (CR), partial responders (PR), stable disease (SD), progressive disease (PD), withdrew consent (WC), and adverse event (AE) . Results of the supervised partial least squares (PLS) test are shown in the top right graph. The lines surrounding each group represent the 95% confidence interval for group separation based on B cell subset analysis. In FIG. 2B, data filtered through the PCA analysis are transformed and shown as a heat map. Healthy and iNHL patient best response to therapy are color coded as indicated, and shown at the top of the graph for each patient. The color range is also shown, representing relative frequencies of the B cell subsets are indicated on the right of the graph. De-identified patient numbers are listed at the bottom of the graph. Groups 1, 2, and 3 are indicated based on the clustering of B cell subset frequencies.

FIG. 3 includes graphs showing that separation of Group 1 is driven primarily by over-representation of transitional B cells. Percent lymphocytes of live cells (top left), percent B cells of live cells (top right), percent transitional B cells of total B cells (bottom left), and percent transitional B cells of naive B cells (bottom right) are shown. The y-axis depicts the frequency of the population, while the x-axis healthy control (HC), and Groups 1, 2, and 3 (G1, G2, and G3, respectively). Each symbol represents an individual healthy volunteer or iNHL patient, with best response to therapy indicated, including complete responders (CR), partial responders (PR, stable disease (SD), progressive disease (PD), withdrew consent (WC), and adverse event (AE). Statistical significance (Wilcoxon) for each patient group relative to HC is shown in each graph.

FIG. 4 includes plots showing over-representation of transitional B cells in Group 1 patients. The FACS plots shown are three examples each of healthy control (top row), Group 1 iNHL patients (middle row), and Group 2 iNHL patients (bottom row). CD27 by IgD demonstrates normal distribution of double negative, naïve, switched memory, and non-switched memory B cell subsets, which are skewed to naive B cells in Groups 1 and 2. The CD27 by CD10 plots demonstrate the increase in transitional (CD10 positive) B cell frequency of the naïve B cells in Group 1 relative to healthy or Group 2.

FIGS. 5A-5B include plots showing that cerdulatinib induces loss of transitional B cell phenotype leading to corresponding increase in the non-transitional naïve B cell compartment over time. In FIG. 5A, plots in which percent B cells of lymphocytes (top left), percent naïve of total B cells (top right), percent transitional of total B cells (bottom left), and percent naïve (excluding transitional) of total B cells (bottom right) are shown. The y-axis depicts the frequency of the B cell population, and the x-axis depicts healthy control (HC) relative to iNHL patient values at baseline (cycle 1; C1) and cycles 2, 3, and 6 (C2, C3, and C6, respectively). Each symbol represents an individual healthy volunteer or iNHL patient, with best response to therapy indicated, including complete responders (CR) partial responders (PR), stable disease (SD), progressive disease (PD), withdrew consent (WC), and adverse event (AE). Statistical significance (Wilcoxon) for each cycle of therapy relative to HC is shown in each graph. In FIG. 5B, plots show representative example from a single Group 1 patient with high baseline transitional B cell frequency of total B cells. FACS plots show CD27 by IgD (top row), CD27 by CD10 (middle row), and CD38 by CD10 (bottom row) at baseline and over time, as indicated by the cycle number.

FIG. 6 show plots demonstrating that separation of Group 3 is mainly driven by over-representation of switched memory and double negative B cells, corresponding with loss of naïve B cells. Percent B cells of lymphocytes (top left), percent naïve of total B cells (top right), percent switched memory of total B cells (bottom left), and percent double negative of total B cells (bottom right) are shown. The y-axis depicts the frequency of the population, while the x-axis healthy control (HC), and Groups 1, 2, and 3 (G1, G2, and G3, respectively). Each symbol represents an individual healthy volunteer or iNHL patient, with best response to therapy indicated, including complete responders (CR), partial responders (PR), stable disease (SD), progressive disease (PD), withdrew consent (WC), and adverse event (AE). Statistical significance (Wilcoxon) for each patient group relative to HC is shown in each graph.

FIG. 7 include plots showing skewing of switched memory and double negative B cell subsets in Group 3 iNHL patients. The representative FACS plots demonstrate the total live B cell populations (Aqua Viability by CD19) and double negative, naïve, switched memory, and non-switched memory (CD27 by IgD) B cell subsets in healthy (top row), and in Group 2 (middle row) and Group 3 (bottom row) patients.

FIG. 8 includes data showing that cerdulatinib does not impact switched memory or double negative B cell populations over time. Percent switched memory of total B cells (left) and percent double negative of total B cells (right) are shown. The y-axis depicts the frequency of the B cell population, and the x-axis depicts healthy control (HC) relative to iNHL patient values at baseline (cycle 1; C1) and cycles 2, 3, and 6 (C2, C3, and C6, respectively). Each symbol represents an individual healthy volunteer or iNHL patient, with best response to therapy, including complete responders (CR), partial responders (PR), stable disease (SD), progressive disease (PD), withdrew consent (WC), and adverse event (AE). Statistical significance (Wilcoxon) for each cycle of therapy relative to HC is shown in each graph.

### DETAILED DESCRIPTION

### 1. Definitions

Unless defined otherwise, all technical and scientific terms used herein have the same meaning as commonly understood by one of ordinary skill in the art to which this disclosure belongs.

As used herein the following terms have the following meanings. Also as used herein and in the appended claims, the singular forms “a”, “an”, and “the” include plural referents unless the context clearly dictates otherwise. Thus, for example, reference to “an agent” includes a plurality of agents.

The term “about” when used before a numerical designation, e.g., temperature, time, amount, and concentration, including range, indicates approximations which may vary by (+) or (-) 10%, 5% or 1%.

As used herein, an “inhibitor” refers to an agent or molecule that inhibits or binds to, partially or totally blocks stimulation or activity, decreases, closes, prevents, delays activation or enzymatic activity, inactivates, desensitizes, or down regulates the activity of a receptor.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “treat,” “treating,” “treatment,” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments as described herein may be applied preventively, prophylactically, palliatively or remedially.

The terms “prevent,” “preventing,” “prevention,” and grammatical variations thereof as used herein, refers to a method of partially or completely delaying or precluding the onset or recurrence of a disorder or condition and/or one or more of its attendant symptoms or barring a subject from acquiring or reacquiring a disorder or condition or reducing a subject’s risk of acquiring or requiring a disorder or condition or one or more of its attendant symptoms.

As used herein, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, “daily dose” refers to a total amount of a therapeutic substance that is to be taken within 24 hours.

Abbreviations:

| | |
|---|---|
| AE | adverse event |
| CR | complete response |
| ORR | overall response rate |
| PD | Progressive disease |
| PR | partial response |
| Pts | patients |
| r/r | relapsed/refractory |
| SD | stable disease |
| SSCₘᵢₙ | steady state plasma minimum concentration |
| WC | withdraw consent |

### 2. Treatments

Disclosed herein are compositions and methods for treating patients having lymphoma, such as B cell lymphoma.

B cell lymphomas are types of lymphomas affecting B cells, which can be either indolent (slow-growing) or aggressive (fast-growing). Many B cell lymphomas are non-Hodgkin lymphomas. Non-limiting examples of B cell lymphomas include Burkitt lymphoma, chronic lymphocytic leukemia/small lymphocytic lymphoma (CLL/SLL), diffuse large B cell lymphoma (DLBCL, including but not limited, to primary mediastinal B cell lymphoma), follicular lymphoma, and mantle cell lymphoma (MCL), marginal zone lymphomas (including but not limited to extranodal marginal zone B cell lymphoma, nodal marginal zone B cell lymphoma, and splenic marginal zone B cell lymphoma), lymphoplasmacytic lymphoma (Waldenstrom macroglobulinemia), and hairy cell leukemia.

As demonstrated in the experimental examples, phenotyping of circulating B cell subpopulations can be used in predicting and/or determining patient outcome to kinase inhibitors for treating B cell lymphomas. Composition of B cell subpopulations (e.g., cell counts for total B cells, transitional B cells, naïve B cells, double negative B cells, switched memory B cells, or any combination thereof, or a ratio between two of these B cell types) can be determined in a patient suffering from a B cell lymphoma.

Striking differences in B cell subset frequencies between healthy and relapsed/refractory (r/r) iNHL patients were observed. For instance, as shown in Example 1, three groups of patients with different overall response rates to cerdulatinib were identified, Groups 1, 2, and 3. Their respective response rates were 22%, 81%, and 46%. The median time on drug (in weeks) for these groups was 8, 59, and 16, respectively. Further, 78% of Group 1 patients discontinued therapy due to adverse event, compared to 13% and 23% for Groups 2 and 3, respectively.

In terms of B cell subset frequencies, relative to healthy, Group 1 patients at baseline exhibited high naïve B cell content, of which there was observed an overrepresentation of CD10+ transitional B cells. Group 2 patients at baseline also exhibited high naïve B cell content, but with normal frequency of transitional B cells. Group 3 patients at baseline exhibited low naïve B cell content relative to healthy and high frequency of switched memory (skewed toward IgM over IgG) or double negative B cells. (Table A.) These data demonstrate that circulating B cell phenotype assessment can predict clinical response, both safety and efficacy, to treatments with kinase inhibitors.

TABLE A

Summary of Changes at Baseline (relative to healthy subjects) & Clinical Observations

| Frequency | Group 1 | Group 2 | Group 3 |
|---|---|---|---|
| Naïve B cells / Total B cells | Increased | Increased | Decreased |
| CD10+ transitional B cells / Naïve B cells or CD10+ transitional B cells / Total B cells | Increased | Unchanged or slightly decreased within naïve B cells | Unchanged |
| Switched memory B cells (IgM) / Total B cells | Unchanged or slightly decreased | Unchanged or slightly decreased | Increased |
| Double negative B cells / Total B cells | Unchanged or slightly decreased | Unchanged or slightly decreased | Increased |
| Response rate | 22% | 81% | 46% |
| Discontinuation rate due to adverse events | 78% | 13% | 23% |

Disclosed herein, in one embodiment, is a method for treating B cell lymphoma. The method, in some embodiments, entails administering a kinase inhibitor to a patient suffering from the B cell lymphoma and is identified to likely respond to the kinase inhibitor.

In some embodiments, a B cell lymphoma patient that is likely to respond to the kinase inhibitor has one or more of the following B cell subset distribution characteristics at baseline (i.e., before the treatment begins), (a) increased naive B cell frequency (or proportion, percentage) within total B cells and unchanged transitional (in particular CD10+) B cell frequencies within total B cells (or slightly decreased within naive B cells), or (b) decreased naive B cell frequencies within total B cells and increased switched memory (skewed toward IgM over IgG) and/or double negative B cells within total B cells.

The terms “increased,” “decreased,” and “unchanged” as used herein, are with reference to suitable controls. When referring to the frequencies of certain B cell subtypes in a B cell lymphoma patient, a suitable control is an individual not having B cell lymphoma. Such an individual is preferably of the same gender as and has similar other characteristics (e.g., age, body weight, race etc) to the patient. The control individual is preferably healthy, but can also be one having a disease or condition that is not B cell lymphoma. In some embodiments, the control individual is not a specific individual, but rather a virtual individual with cell counts pooled/averaged from a group of control individuals. These terms are sometimes substituted with more conventional terms such as “overrepresentation,” “underrepresentation,” and “normal representation”, or simply “high,” “low,” and “normal.”

In some embodiments, the identification of a suitable patient does not require comparison of the B cell subtype populations to controls. Instead, based on known data or conventional knowledge, a predetermined threshold or range can be used to assess whether a number is increased, decreased or unchanged.

For instance, with respect to the frequency of translational B cells within total B cells, higher than 25% is considered increased (or high) as compared to controls. Alternatively, in some embodiments, higher than 15%, 20%, 30%, 35%, 40%, 45% or 50% is considered increased (or high) as compared to controls.

With respect to the frequency of translational B cells within naive B cells, in some embodiments, higher than 30% is considered increased (or high) as compared to controls. Alternatively, in some embodiments, higher than 15%, 20%, 25%, 35%, 40%, 45% or 50% is considered increased (or high) as compared to controls.

With respect to the frequency of naive B cells within total B cells, in some embodiments, higher than 30%, or alternatively higher than 40%, 35%, 25%, 20%, 15%, 10%, or 5% is considered increased (or high) as compared to controls. In some embodiments, lower than 30%, or alternatively lower than 40%, 35%, 25%, 20%, 15%, 10%, or 5% is considered decreased (or low) as compared to controls.

With respect to the frequency of switched memory B cells within total B cells, in some embodiments, higher than 25%, 30%, 35%, 40%, 45%, 50%, 55%, 60%, 70%, 80% or 90% is considered increased (or high) as compared to controls. In some embodiments, lower than 25%, 30%, 35%, 40%, 45%, 50%, 55%, 60%, 70%, 80% or 90% is considered decreased (or low) as compared to controls.

With respect to the frequency of double negative B cells within total B cells, in some embodiments, higher than 25%, 30%, 35%, 40%, 45%, 50%, 55%, 60%, 70%, 80% or 90% is considered increased (or high) as compared to controls. In some embodiments, lower than 25%, 30%, 35%, 40%, 45%, 50%, 55%, 60%, 70%, 80% or 90% is considered decreased (or low) as compared to controls.

In some embodiments, the kinase inhibitor used in the methods disclosed herein for treating B cell lymphomas is cerdulatinib, which is a dual spleen tyrosine kinase (SYK) and Janus kinase (JAK) inhibitor.

The patient can be, for example, an iNHL patient (e.g., r/r iNHL patient). The patient can be a patient that is being considered for the kinase inhibitor treatment, a patient is to receive the kinase inhibitor treatment, or a patient that is under the kinase inhibitor treatment. The patient can be a patient that is treatment naïve, or one that has been treated or is being treated with a B cell lymphoma treatment other than the kinase inhibitor treatment.

The patient can also have other characteristics in addition to the composition of B cell subpopulations disclosed herein. For example, the patient has not suffered from a diabetic or pre-diabetic condition prior to the administration of the kinase inhibitor. In some embodiments, the patient has had fewer than 5 prior treatments for the B cell lymphoma.

In some embodiments, the method can further comprise providing a B cell lymphoma treatment other than the kinase inhibitor treatment. For example, the method can comprise administering to the patient an additional kinase inhibitor, an antibody therapeutic for treating B cell lymphoma, a chemotherapeutic agent, or both.

Kinase inhibitors include, but are not limited to, inhibitors for the phosphoinositide 3-kinase (PI3K), inhibitors for Spleen tyrosine kinase (SYK), inhibitors for Janus kinase (JAK), and inhibitors for Bruton’s tyrosine kinase (BTK). Despite recent advances, there still needs for well tolerated and efficacious therapeutic options for relapsed/refractory (r/r) patients, and methods for determining and/or predicting the responsiveness of a patient to the therapeutic agents for treating B cell lymphomas.

Phosphoinositide 3-kinase (PI3K) inhibitors can be used to treat B cell lymphoma. PI3K inhibitors include, for example, PI3Kα inhibitors, PI3Kδ inhibitors and PI3Kγ inhibitors. Non-limiting examples of PI3K inhibitor include idelalisib (a PI3Kδ inhibitor), copanlisib (predominantly against PI3Kα and PI3Kδ), duvelisib (IPI-145, a dual inhibitor of PI3Kδ and PI3Kγ), AMG-319 (a PI3Kδ inhibitor, clinical trial no. NCT01300026), umbralisib (TGR-1202, a PI3Kδ inhibitor), voxtalisib (SAR245409, XL765), and BKM120 (an inhibitor for all four class I PI3K isoforms, clinical trial no. NCT01719250). In some embodiments, the PI3K inhibitor is used in combination with one or more additional therapeutic agents for treating a B cell lymphoma. The one or more additional therapeutic agents can, for example, comprise ofatumumab, rituximab, bendamustine, GS-9973, ublituximab, obinutuzumab, chlorambucil, or any combination thereof.

Spleen tyrosine kinase (SYK) is a cytosolic non-receptor protein tyrosine kinase (PTK) and is mainly expressed in hematopoietic cells. SYK was recognized as a critical element in the B cell receptor signaling pathway, and also a key component in signal transduction from other immune receptors like Fc receptors and adhesion receptors. Non-limiting examples of SYK inhibitors include fostamatinib (previously known as R788), entospletinib (GS-9973), cerdulatinib, and TAK-659. In some embodiments, the SYK inhibitor is used in combination with one or more additional therapeutic agents for treating B cell lymphoma. The one or more additional therapeutic agents can, for example, comprise idelalisib, vincristine, dexamethasone, or any combination thereof.

Janus kinase (JAK) inhibitors can also be used to treat B cell lymphoma. An JAK inhibitor can inhibit the activity of one or more of the Janus kinase family of enzymes (JAK1, JAK2, JAK3, TYK2), thereby interfering with the JAK-STAT signaling pathway. In some embodiments, the JAK inhibitor is a JAK3 inhibitor. In some embodiments, the JAK inhibitor is a JAK2 inhibitor. In some embodiments, the JAK inhibitor is a JAK⅟JAK2 inhibitor. Non-limiting examples of the JAK inhibitor include ruxolitinib, fedratinib, pacritinib, and cerdulatinib.

The Bruton tyrosine kinase (BTK) is a central hub in the B cell receptor (BCR) pathway and strongly influences B cell maturation, differentiation and proliferation. The kinase inhibitor used in the methods disclosed herein can be a BTK inhibitor. Non-limiting example of BTK inhibitors include ibrutinib, acalabrutinib (ACP-196, NCT02029443), CC-292 (AVL-292), ONO/GS-4059 (NCT01659255), and BGB-3111. In some embodiments, the BTK inhibitor is used in combination with one or more additional therapeutic agents for treating a B cell lymphoma. The one or more additional therapeutic agents can, for example, comprise rituximab, bendamustine, or any combination thereof.

### 3. Patient Screening and Monitoring

Methods are provided for identifying patients with B cell malignancies that will or will not respond to a kinase inhibitor of the present disclosure (e.g., cerdulatinib). Patients can be identified by flow cytometric analysis of baseline whole blood sample as having high naïve B cell frequencies with a transitional (CD10+) B cell phenotype in the total B cell population.

Methods are also provided for identifying patients suitable for the treatment of the present disclosure, including methods for determining in a blood sample derived from the patient, the cell count of total B cells and/or different B cell subpopulations. For example, the method can include determining/counting in a blood sample from the patient, total B cells, transitional B cells, naive B cells, double negative B cells, switched memory B cells, live cells, or any combination thereof. In some embodiments, the method further comprises obtaining the blood sample from the patient.

Total B cells can include transitional B cells, naïve B cells, double negative B cells, non-switched memory cells, and switched memory B cells. CD 19 is a marker for all B cells. Transitional B cells are B cells at an intermediate stage in their development between bone marrow immature B cells and mature B cells in circulating blood and secondary lymphoid organs. Transitional B cells have high expression levels of CD24, CD38 and CD10. Naive B cells are B cells that have not been exposed to an antigen. Once exposed to an antigen, a naive B cell either becomes a memory B cell or a plasma cell. Double negative B cells are B cells that have undergone class switching but lack expression of immunoglobulin D and the memory marker CD27. CD27 is a marker for the memory subset of cells (including switched memory (SW) and non-switched memory (NSM) B cells). B cells that do not express IgD have undergone class-switching and express IgG, IgA, or IgE. CD19+CD27+IgD- cells can be referred to as switched memory B cells and are indicators of normal B-cell activation and development in germinal centers in lymph nodes or other secondary lymphoid tissues. CD19+CD27+IgD+ cells can be referred to as non-switched memory B cells.

In some embodiments, B cells are identified based on the CD 19 expression. In some embodiments, IgD and CD27 expression is used to delineated double negative (DN), naïve, switched memory (SM), and non-switched memory (NSM) B cells. Double negative B cells are CD27-IgD-. Naive B cells are CD27-IgD+. Switched memory B cells are CD27+IgD-. Non-switched memory B cells are CD27+IgD+. In some embodiments, transitional B cells of naive are identified based on the high CD 10 expression. In some embodiments, IgG and IgM switched memory B cells are identified based on the high IgG expression and the high IgM expression, respectively. In some embodiments, plasma cells are identified based on high CD27 expression and CD 138.

In some embodiments, the expression levels of protein markers on the B cells (e.g., CD19, CD20, HLA-DR, CD27, IgD, CD10, CD38, CD138, IgG, and IgM) are determined by staining cells with antibodies (e.g., monoclonal antibodies) that are conjugated with different identifiers (e.g., fluorophores or barcodes) and specifically bind to these protein markers. The expression levels of the protein markers on the stained cells can be determined flow cytometrically based on the intensities of the fluorophores conjugated to the different antibodies. The expression levels of the protein markers on the stained cells can be determined by quantifying (e.g., by PCR) the barcodes of different sequences conjugated to the different antibodies. In some embodiments, the expression levels of protein markers on B cells are determined based on the expression levels of the corresponding mRNAs encoding the protein markers. CD19, CD20, HLA-DR are markers for B cells. CD27 is a marker for switched memory B cells. IgD is a marker for naive B cells. CD10 is a marker for transitional B cells of naive. CD38 and CD138 are markers for plasma B cells. IgG and IgM are markers for IgG and IgM switched memory B cells, respectively.

Also disclosed herein include a method for monitoring the treatment of a B-cell lymphoma patient. The method can comprise, for example, determining, in a blood sample isolated from a patient receiving a kinase inhibitor treatment, the transitional B cells, wherein an increase of the transitional B cells, as compared to prior to the administration of the kinase inhibitor, indicates that the patient is likely not responsive to or suffers adverse effects from the treatment of the kinase inhibitor. The method can further comprise administering to the patient the kinase inhibitor or a pharmaceutically acceptable salt thereof, before, during, or after determining the transitional B cells in the patient. The increase of the transition B cells can, for example, indicate that the patient is not responsive, or has a more than 5%, 10%, 15%, 20%, 25%, 30%, 35%, 40%, 45%, 50%, 55%, 60%, 65%, 70%, 75%, or more, or a number or a range between any two of these values, likelihood not to be responsive to the treatment of the kinase inhibitor. The increase of the transition B cells can, in some embodiments, indicate that the patient has a more than 5%, 10%, 15%, 20%, 25%, 30%, 35%, 40%, 45%, 50%, 55%, 60%, 65%, 70%, 75%, or more, or a number or a range between any two of these values, likelihood to suffer one or more adverse effects from the treatment of the kinase inhibitor. The increase of the transition B cells can, in some embodiments, indicate that the patient has a more than 5%, 10%, 15%, 20%, 25%, 30%, 35%, 40%, 45%, 50%, 55%, 60%, 65%, 70%, 75%, or more, or a number or a range between any two of these values, likelihood to suffer a larger extent from one or more adverse effects from the treatment of the kinase inhibitor as compared to the patients who do not have an increase of the transitional B cells as compared to prior to the administration of the kinase inhibitor.

The increase of the transitional B cells is, in some embodiments, an increase of a ratio of the transitional B cells over one or more of total B cells, total lymphocytes, and total live cells. The transitional B cells can comprise CD10+ transitional B cells, or CD38+ transitional B cells, or both.

The method can comprise, in some embodiments, ceasing the administration of the kinase inhibitor or the pharmaceutically acceptable salt thereof to the patient.

Also disclosed herein is a method for identifying a patient as suitable for treatment with a kinase inhibitor. The method can comprise determining, in a blood sample derived from a B-cell lymphoma patient, total B cells, transitional B cells, naive B cells, and any combination thereof, wherein the patient is identified as suitable for the treatment based on the B cell subset frequencies at baseline, as discussed above.

The patient can also have other characteristics in addition to the composition of B cell subpopulations disclosed herein. For example, in some embodiments, the patient does not suffer from a diabetic or pre-diabetic condition prior to the administration. In some embodiments, the patient is treatment naive. In some embodiments, the patient has had fewer than 5 prior treatments for the B-cell lymphoma.

All the B-cell lymphomas are contemplated herein. For example, the B-cell lymphoma can be a non-Hodgkin lymphoma (NHL), for example, indolent NHL (iNHL). In some embodiments, the method further comprises administering to the patient an additional kinase inhibitor, a chemotherapeutic agent, or both.

The cell counts, for example the counts for total B cells, transitional B cells, naive B cells, double negative B cells, and/or switched memory B cells, can be made in a blood sample obtained from the patient.

The method can further comprise determining in a blood sample derived from the patient, the cell count of total B cells and/or different B cell subpopulations. In some embodiments, the method further comprises determining in a blood sample from the patient, total B cells, transitional B cells, naive B cells, double negative B cells, switched memory B cells, or any combination thereof.

The method can further comprise determining in a blood sample derived from the patient, the cell count of total B cells and/or one or more of different B cell subpopulations. For example, the method can further comprise determining/counting in a blood sample from the patient, total B cells, transitional B cells, naive B cells, double negative B cells, switched memory B cells, live cells, or any combination thereof. In some embodiments, the method further comprises obtaining the blood sample from the patient.

As described herein, the cell counts, for example the counts for total B cells, transitional B cells, naïve B cells, double negative B cells, and/or switched memory B cells, can be made in a blood sample (or another type of sample, such as a tissue sample) derived from the patient. The blood sample can be a sample directly obtained from the patient, or a processed product from a blood sample directly obtained from the patient. The cell counts can be, for example, an absolute count of a B-cell type of interest, or a count of a B-cell type of interest relative to other cell types (e.g., total live cells), another B-cell type (e.g., naïve B cells) or a combination of B-cell types (e.g., total B cells). Methods for obtaining cell counts are well known in the art, including histochemistry stain, and flow cytometry. In some embodiments, obtaining cell counts can comprise using cell surface markers specific for the cell type of interest. For example, obtaining cell counts for transitional B cells can comprise using CD10, or CD38, or both to identify transition B cells. Examples of transitional B cells include, but are not limited to, CD10+ transitional B cells, or CD38+ transitional B cells, or both.

In the methods described herein, the kinase inhibitor can be, for example, a PI3K inhibitor, a SYK inhibitor, a Jak inhibitor, a BTK inhibitor, or a combination thereof. Examples of the PI3K inhibitor include, but are not limited to, idelalisib, IPI-145, AMG-319, TGR-1202, and BKM120. Examples of the SYK inhibitor include, but are not limited to, fostamatinib, entospletinib, cerdulatinib, and TAK-659. Examples of the Jak inhibitor include, but are not limited to, ruxolitinib, fedratinib, and cerdulatinib. Non-limiting examples of the BTK inhibitor include ibrutinib, acalabrutinib, CC-292 (AVL-292), ONO/GS-4059, and BGB-3111. In some embodiments, the kinase inhibitor is cerdulatinib.

In some embodiments, the patient was previously administered a drug selected from the group consisting of an alkylating agent, an anti-CD20 antibody, a BCL-2 inhibitor, a BTK inhibitor, a P13Kδ inhibitor, a platinum-based drug, an antimetabolite, an anthracycline, a BCR pathway inhibitor, and other agents used for treating a hematologic cancer. In some embodiments, the drug is rituximab, ibrutinib, idelalisib, tofacitinib, fludararbine (fludarabine phosphate, FLUDARA®), or ABT-199 (venetoclax). In some embodiments, the drug is R-CHOP (rituximab; cyclophosphamide; doxorubicin hydrochloride; (vincristine); prednisone). In some embodiments, the drug is R-CVP (rituximab; cyclophosphamide; vincristine; prednisone). In some embodiments, the drug is bevacizumab. In some embodiments, the drug is a combination of fludarabine and rituximab, a combination of bendamustine and rituximab, or a combination of bevacizumab and rituximab.

In some embodiments of the methods described herein, the patient has resistance to a drug, which is not a kinase inhibitor (e.g., cerdulatinib). Non-limiting examples of these drugs are an anti-CD20 antibody, a BCL-2 inhibitor, a BTK inhibitor, a P13Kδ inhibitor, rituximab, a platinum-based drug, an antimetabolite, ibrutinib, idelalisib, fludararbine (fludarabine phosphate, FLUDARA®), anthracyclines, a BCR pathway inhibitor, ABT-199 (venetoclax), tofacitinib, or another chemotherapeutic agent used for treating a hematologic cancer. In some embodiments, the patient has resistance to a drug selected from the group consisting of ibrutinib, idelalisib, tofacitinib, fludararbine (fludarabine phosphate, FLUDARA®), or ABT-199 (venetoclax). In some embodiments, the patient has resistance to ibrutinib. Other non-limiting examples of chemotherapeutic agents include alkylating agents, cytoskeletal disruptors, epothiolones, histone deacetylase inhibitors, inhibitors of topoisomerase I, inhibitors of topoisomerase II, nucleotide analogs and precursor analogs, antibiotics, retinoids, vinca alkaloids, retinoids, vinca alkaloids, or a combination thereof.

Some chemotherapeutic agents suffer from drug resistance in a patient, for example, due to BCR IL-4 mediated signaling and/or BCR activation pathways, which are protective of hematological cancer. According to embodiments of the present disclosure, cerdulatinib can overcome these protective mechanisms, which lead to drug resistance. The patient in the methods disclosed herein can be a patient exhibiting drug resistance to, and/or a relapsed for, of a hematological cancer for various reasons. For example, the patient may have a mutation linked to relapse and/or a resistance to a drug for treating a hematological cancer. For example, the patient may have a del17p mutation, a P53 mutation, an ATM mutation, a STAT mutation, a STAT6 mutation, a C481S STAT6 mutation, a mutation associated with the NOTCH pathway, or a mutation associated with the Caderin pathway. In some embodiments, the patient may have a S86A mutation in STAT. In some embodiments, the patient may have a dell7p mutation, del11q mutation, a P53 mutation, an ATM mutation, a STAT mutation, a STAT 6 mutation, a C481S STAT6 mutation, a mutation associated with the NOTCH pathway, a mutation associated with the Caderin pathway, or a combination thereof. In some embodiments, the patient does not have a mutation in one or more of P53, BTK, and EP300 gene. In some embodiments, the patient has a MYD88 mutation, a CARD 11 mutation, an A20 mutation, or any combination thereof. In some embodiments, the patient has high-risk genetic abnormalities including del11q, trisomy 12, and del17p. In some embodiments, the patient has a del17p mutation. In some embodiments, the patient has a del11q mutation. In some embodiments, the patient has a BTK mutation. In some embodiments, the patient have a poor prognosis such as unmutated IGHV, high CD49d, ZAP-70, surface IgM expression, or any combination thereof.

In certain embodiments, the patient is 60 years or older and relapsed after a first line cancer therapy. In certain embodiments, the patient is 18 years or older and is relapsed or refractory after a second line cancer therapy. In certain embodiments, the patient is 60 years or older and is primary refractory to a first line cancer therapy. In certain embodiments, the patient is 70 years or older and is previously untreated. In certain embodiments, the patient is 70 years or older and is ineligible and/or unlikely to benefit from cancer therapy.

### 4. Dosages

The dosage regime of the kinase inhibitor (e.g., cerdulatinib) can be determined by the condition of the B cell lymphoma patient, for example the B cell subpopulations of the patient (e.g., a human patient). The methods disclosed herein can comprise administering to the patient an effective amount of a kinase inhibitor (e.g., cerdulatinib), or a pharmaceutically acceptable salt thereof, or a pharmaceutical composition. For example, the method can comprise administering the patient a daily dose of, or about, 5 mg, 8 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, or more, or a number or a range between any two of these values, the kinase inhibitor (e.g., cerdulatinib), or a pharmaceutically acceptable salt thereof, and at least one pharmaceutically acceptable carrier or excipient. In some embodiments, the method comprises administering the patient a daily dose of, about 10 mg to about 75 mg of cerdulatinib, or a pharmaceutically acceptable salt thereof, and at least one pharmaceutically acceptable excipient or carrier. In some embodiments, the method comprises administering the patient a daily dose of, about 30 mg to about 80 mg of cerdulatinib, or a pharmaceutically acceptable salt thereof, and at least one pharmaceutically acceptable excipient or carrier.

The patient, for example, can have a relapsed or refractory B cell lymphoma. For example, the method can comprise administering to the patient (e.g., a patient having a relapsed or refractory B cell lymphoma) a twice daily a dose of, or about, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, or more, or a number or a range between any two of these values, of the kinase inhibitor (e.g., cerdulatinib or a pharmaceutically acceptable salt thereof). In some embodiments, the patient is administered with a twice daily a dose of, or about, 35 mg or 40 mg of the kinase inhibitor (e.g., cerdulatinib or a pharmaceutically acceptable salt thereof).

In some embodiments, the method can comprise administering to the patient (e.g., a patient having a relapsed or refractory B cell lymphoma) a daily dose of, or about, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, or more, or a number or a range between any two of these values, of the kinase inhibitor (e.g., cerdulatinib or a pharmaceutically acceptable salt thereof). The daily dose can be given to the patient via a single or multiple administrations. In some embodiments, the patient is administered with a daily dose of, or about, 10 mg to 75 mg of the kinase inhibitor (e.g., cerdulatinib or a pharmaceutically acceptable salt thereof). In some embodiments, the patient is administered with a daily dose of, or about, 25 mg to 45 mg of the kinase inhibitor (e.g., cerdulatinib or a pharmaceutically acceptable salt thereof). In some embodiments, the daily dose of the kinase inhibitor (e.g., cerdulatinib, or a pharmaceutically acceptable salt thereof) is about 15 mg, 30 mg, 45 mg, or 50 mg. In some embodiments, the daily dose of the kinase inhibitor (e.g., cerdulatinib, or a pharmaceutically acceptable salt thereof) is about 90 mg. In some embodiments, the daily dose of the kinase inhibitor (e.g., cerdulatinib, or a pharmaceutically acceptable salt thereof) is about 70 mg. In some embodiments, the daily dose of the kinase inhibitor (e.g., cerdulatinib, or a pharmaceutically acceptable salt thereof) is administered twice daily at about 35 mg per dose.

In some embodiments, the administration of the kinase inhibitor (e.g., cerdulatinib, or a pharmaceutically acceptable salt thereof) is once daily. In some embodiments, the administration is twice daily. In some embodiments, the administration is three times daily.

In some embodiments, the therapeutically effective amount of the kinase inhibitor (e.g., cerdulatinib, or a pharmaceutically acceptable salt thereof) used in the methods provided herein is at least about 10 mg per day. For example, the therapeutically effective amount of the kinase inhibitor (e.g., cerdulatinib, or a pharmaceutically acceptable salt thereof) can be at least about 10, 20, 30, 40, or 50 mg per dosage. In one embodiment, the therapeutically effective amount of the kinase inhibitor (e.g., cerdulatinib, or a pharmaceutically acceptable salt thereof) is at least about 10, 20, 30, 40, 50, 60, 70, 80, 90 or 100 mg per day.

In some embodiment, the therapeutically effective amount of the kinase inhibitor (e.g., cerdulatinib or a pharmaceutically acceptable salt thereof) is at least 30 mg, 35 mg, 40 mg, 45 mg, 50 mg, 55 mg, 60 mg, or 65 mg per day. In some embodiments, the therapeutically effective amount of the kinase inhibitor (e.g., cerdulatinib, or a pharmaceutically acceptable salt thereof) is at least about 15 mg, 20 mg, 25 mg, 30 mg, or 35 mg and is administered twice daily.

In some embodiments, the therapeutically effective amount of the kinase inhibitor (e.g., cerdulatinib, or a pharmaceutically acceptable salt thereof) is no more than about 500, 400, 300, 200, 150, 120, or 100 mg per day. In one embodiment, the therapeutically effective amount of the kinase inhibitor (e.g., cerdulatinib, or a pharmaceutically acceptable salt thereof) is no more than about 300, 200, 150, 120, 100, 90, 80, 70, 60, 55 or 50 mg per dosage.

In some embodiments, the therapeutically effective amount of the kinase inhibitor (e.g., cerdulatinib or a pharmaceutically acceptable salt thereof) is no more than about 100 mg, 95 mg, 90 mg, 85 mg, 80 mg, or 75 mg per day. In some embodiments, the therapeutically effective amount of the kinase inhibitor (e.g., cerdulatinib, or a pharmaceutically acceptable salt thereof) is no more than 45 mg, 40 mg, 35 mg, or 30 mg and is administered twice daily.

In some embodiments, the therapeutically effective amount of the kinase inhibitor (e.g., cerdulatinib or a pharmaceutically acceptable salt thereof), whether alone or in combination with another therapeutic agent, is administered at from about 10 mg to 200 mg, from about 25 mg to 150 mg, from about 50 to 120 mg, from about 80 to 100 mg a day. In some embodiments, the therapeutically effective amount of the kinase inhibitor (e.g., cerdulatinib or a pharmaceutically acceptable salt thereof), whether alone or in combination with another agent, is 25 mg to 120 mg daily. In some embodiments, the effective amount of cerdulatinib is 25 mg to 50 mg twice daily. In some embodiment, the therapeutically effective amount cerdulatinib, whether alone or in combination with another agent, is administered at from about 10 mg to 150 mg, from about 25 mg to 120 mg, from about 15 mg to about 40 mg, from about 30 mg to about 40 mg, from about 40 mg to about 50 mg, from about 30 to 80 mg, from about 40 to 50 mg a dosage, once or twice a day. In some embodiments, the kinase inhibitor (e.g., cerdulatinib or a pharmaceutically acceptable salt thereof), whether alone or in combination with another agent, is administered once, twice, three times, or four times a day.

The patient may have relapsed or not responded to one or more prior treatments for B cell lymphoma, for example chemotherapies. In some embodiments, the patient has relapsed or not responded to a prior treatment for B cell lymphoma. In some embodiments, the patient has failed at least two prior treatments for B cell lymphoma. In some embodiments, the patient has failed at least one prior treatments for B cell lymphoma. It can be advantageous to treat a patient that has had fewer than a desirable number of prior treatment for the B cell lymphoma. For example, the patient can be a patient has had no more than, or fewer than, 7, 6, 5, 4, 3, 2, 1, or a number or a range between any two of these values, prior treatments for the B cell lymphoma or other types of B cell lymphomas. In some embodiments, the patient has had fewer than 5 prior treatments for the B cell lymphoma, or other types of B cell lymphoma.

The specific amount of a kinase inhibitor (e.g., cerdulatinib) described herein refers to the amount of the kinase inhibitor free base, such as the cerdulatinib free base, i.e., the compound of formula I. However, it is understood that a pharmaceutically acceptable salt, co-crystal or solvate of the kinase inhibitor or a mixture thereof may be administered in an amount that provides the stated amount of the kinase inhibitor. Examples of pharmaceutically acceptable salts of the kinase inhibitor include those derived from inorganic or organic acids, such as acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, edisylate, fumarate, lucoheptanoate, glycerophosphate, hemisulfate, heptanoate, hexanoate, hydrochloride, bis-hydrochloride, hydrobromide, hydroiodide, 2-hydroxyethanesulfonate, lactate, maleate, methane sulfonate, bis-methanesulfonate, 2-naphthalenesulfonate, naphthalene disulfate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulfates, phosphates, nitrates, sulphamates, malonates, salicylates, methylene-bis-b-hydroxynaphthoates, gentisates, isethionates, di-ptoluoyltartrates, ethanesulphonates, cyclohexylsulphamates, quinates, and the like.

A kinase inhibitor of the present disclosure (e.g., cerdulatinib) or a salt thereof can be administered in unsolvated forms as well as solvated forms, including hydrated forms, or form co-crystals with another compound. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

In some embodiments, the kinase inhibitor is cerdulatinib. Cerdulatinib can be administered as a hydrochloride salt (cerdulatinib HCl). In some embodiments, the cerdulatinib HCl is in a crystalline form. In some embodiments, the cerdulatinib HCl is in a crystalline form characterized by an X-ray powder diffractogram comprising peaks at 8.7, 15.9, and 20.0 °2θ, each ± 0.2 °2θ, as determined on a diffractometer using Cu-Kα radiation (cerdulatinib HCl Form I). In some embodiments, cerdulatinib HCl Form I is further characterized by one or more peaks at 11.5, 22.5, and 25.5 °2θ, each ± 0.2 °2θ. In some embodiments, cerdulatinib HCl Form I is further characterized by a differential scanning calorimetry curve comprising an endotherm with onset at about 288° C.

### 5. Combination Treatments

In one embodiment, the treatment methods can further include administration of an effective amount of another agent, such as a chemotherapeutic agent useful for treating the cancer. In some embodiments, the kinase inhibitor (e.g., cerdulatinib) is co-administered with an effective amount of the another agent or a pharmaceutically acceptable salt, co-crystal or solvate thereof. In some embodiments, the another agent is a chemotherapeutic agent. In some embodiments, the agent is co-administered with the kinase inhibitor simultaneously or sequentially.

A kinase inhibitor of the present disclosure (e.g., cerdulatinib) can be used in combination with one or more other enzyme/protein/receptor inhibitors for the treatment of diseases, such as cancer. Examples of cancers include solid tumors and liquid tumors, such as blood cancers. For example, a kinase inhibitor of the present disclosure (e.g., cerdulatinib) can be combined with one or more inhibitors of the following kinases for the treatment of cancer: Akt1, Akt2, Akt3, TGFβR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In some embodiments, a kinase inhibitor of the present disclosure (e.g., cerdulatinib) can be combined with one or more of the following inhibitors for the treatment of cancer. Non-limiting examples of inhibitors that can be combined with a kinase inhibitor of the present disclosure (e.g., cerdulatinib) for treatment of cancers include an FGFR inhibitor (FGFR1, FGFR2, FGFR3 or FGFR4, e.g., AZD4547, BAY1187982, ARQ087, BGJ398, BIBF1120, TK1258, lucitanib, dovitinib, TAS-120, JNJ-42756493, Debio1347, INCB54828, INCB62079 and INCB63904), a JAK inhibitor (JAK1 and/or JAK2, e.g., ruxolitinib, baricitinib or INCB39110), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor, a CSF1R inhibitor (e.g., PLX3397 and LY3022855), a TAM receptor tyrosine kinases (Tyro-3, Axl, and Mer), an angiogenesis inhibitor, an interleukin receptor inhibitor, bromo and extra terminal family members inhibitors (for example, bromodomain inhibitors or BET inhibitors such as OTX015, CPI-0610, INCB54329 and INCB57643) and an adenosine receptor antagonist or combinations thereof. Inhibitors of HDAC such as panobinostat and vorinostat. Inhibitors of c-Met such as onartumzumab, tivantnib, and INC-280. Inhibitors of BTK such as ibrutinib. Inhibitors of mTOR such as rapamycin, sirolimus, temsirolimus, and everolimus. Inhibitors of Raf, such as vemurafenib and dabrafenib. Inhibitors of MEK such as trametinib, selumetinib and GDC-0973. Inhibitors of Hsp90 (e.g., tanespimycin), cyclin dependent kinases (e.g., palbociclib), PARP (e.g., olaparib) and Pim kinases (LGH447, INCB053914 and SGI-1776) can also be combined with a kinase inhibitor of the present disclosure (e.g., cerdulatinib).

A kinase inhibitor of the present disclosure (e.g., cerdulatinib) can be used in combination with one or more immune checkpoint inhibitors. Exemplary immune checkpoint inhibitors include inhibitors against immune checkpoint molecules such as CD20, CD27, CD28, CD39, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, PD-1, PD-L1, and PD-L2. In some embodiments, the immune checkpoint molecule is a stimulatory checkpoint molecule selected from CD27, CD28, CD40, ICOS, OX40, GITR, and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors, and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab. In some embodiments, the anti PD-1 antibody is SHR-1210.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the inhibitor of an immune checkpoint molecule is an inhibitor of CSF1R, e.g., an anti-CSF1R antibody. In some embodiments, the anti-CSF1R antibody is IMC-CS4 or RG7155.

In some embodiments, the inhibitor of an immune checkpoint molecule is an inhibitor of LAG3, e.g., an anti-LAG3 antibody. In some embodiments, the anti-LAG3 antibody is BMS-986016, LAG525, IMP321, or GSK2831781.

In some embodiments, the inhibitor of an immune checkpoint molecule is an inhibitor of GITR, e.g., an anti-GITR antibody. In some embodiments, the anti-GITR antibody is TRX518, MK-4166, MK1248, BMS-986156, MEDI1873, or GWN323.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562, MEDI6469, MOXR0916, PF-04518600, or GSK3174998. In some embodiments, the OX40L fusion protein is MEDI6383.

In some embodiments, the inhibitor of an immune checkpoint molecule is an inhibitor of TIM3, e.g., an anti-TIM3 antibody. In some embodiments, the anti-TIM3 antibody is MBG-453.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat and NGL919. An example of an arginase inhibitor is CB-1158.

A kinase inhibitor of the present disclosure (e.g., cerdulatinib) can be used in combination with bispecific antibodies. In some embodiments, one of the domains of the bispecific antibody targets PD-1, PD-L1, CTLA-4, GITR, OX40, TIM3, LAG3, CD137, ICOS, CD3, or TGFβ receptor.

A kinase inhibitor of the present disclosure (e.g., cerdulatinib) can be used in combination with one or more agents for the treatment of diseases such as cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX). In some embodiments, the immunomodulatory agent is lenalidomide (LEN), or pomalidomide (POM).

A kinase inhibitor of the present disclosure (e.g., cerdulatinib) can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, oncolytic virotherapy and immunomodulating small molecules, including thalidomide or JAK½ inhibitor and the like. The compounds can be administered in combination with one or more anti-cancer drugs, such as a chemotherapeutics. Examples of chemotherapeutics include any of: abarelix, abiraterone, afatinib, aflibercept, aldesleukin, alemtuzumab, alitretinoin, allopurinol, altretamine, anastrozole, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, oxaliplatin, paclitaxel, pamidronate, panitumumab, pazopanib, pegaspargase, pegfilgrastim, pemetrexed disodium, pentostatin, pilaralisib, pipobroman, plicamycin, ponatinib, prednisone, procarbazine, quinacrine, rasburicase, regorafenib, reloxafine, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vinorelbine, vorinostat, and zoledronate.

Other anti-cancer agent(s) include antibody therapeutics such as trastuzumab (Herceptin®), antibodies to costimulatory molecules such as CTLA-4 (e.g., ipilimumab or tremelimumab), 4-1BB, antibodies to PD-1 and PD-L1, or antibodies to cytokines (IL-10, TGF-.beta., etc.). Examples of antibodies to PD-1 and/or PD-L1 that can be combined with compounds of the present disclosure for the treatment of cancer or infections such as viral, bacteria, fungus and parasite infections include, but are not limited to, nivolumab, pembrolizumab, MPDL3280A, MEDI-4736, and SHR-1210.

Other anti-cancer agents include inhibitors of kinases associated cell proliferative disorder. These kinases include but not limited to Aurora-A, CDK1, CDK2, CDK3, CDK5, CDK7, CDK8, CDK9, ephrin receptor kinases, CHK1, CHK2, SRC, Yes, Fyn, Lck, Fer, Fes, Syk, Itk, Bmx, GSK3, JNK, PAK1, PAK2, PAK3, PAK4, PDK1, PKA, PKC, Rsk, and SGK.

Other anti-cancer agents also include those that block immune cell migration such as antagonists to chemokine receptors, including CCR2 and CCR4.

The kinase inhibitors of the present disclosure (e.g., cerdulatinib) can further be used in combination with one or more anti-inflammatory agents, steroids, immunosuppressants or therapeutic antibodies.

The kinase inhibitors of the present disclosure (e.g., cerdulatinib) can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI, and/or tyrosinase, or tumor cells transfected to express the cytokine GM-CSF.

A kinase inhibitor of the present disclosure (e.g., cerdulatinib) can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV), and Kaposi’s Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

A kinase inhibitor of the present disclosure (e.g., cerdulatinib) can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

A kinase inhibitor of the present disclosure (e.g., cerdulatinib) can be used in combination with bone marrow transplant for the treatment of a variety of tumors of hematopoietic origin.

Suitable antiviral agents contemplated for use in combination with the compounds of the present disclosure can comprise nucleoside and nucleotide reverse transcriptase inhibitors (NRTIs), non-nucleoside reverse transcriptase inhibitors (NNRTIs), protease inhibitors, and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(-)-FTC]; beta-L-FD4 (also called beta-L-D4D and named beta-L-2′,3′-dicleoxy-5-fluoro-cytidene); DAPD, ((-)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimid-inedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside, and Yissum Project No. 11607.

When more than one pharmaceutical agent is administered to a patient, they can be administered simultaneously, separately, sequentially, or in combination (e.g., for more than two agents). In some embodiments, the other agent can be selected from one of the classes detailed below.

Polyfunctional alkylating agents, exemplified by cyclophosphamide (cytoxan), mechlorethamine, melphalan (alkeran), chlorambucil (leukeran), thiopeta (thioplex), and busulfan (myleran);
Alkylating drugs, exemplified by procarbazine (matulane), dacarbazine (dtic), altretamine (hexalen), clorambucil, cisplatin (platinol), carboplatin, ifosafamide, and oxaliplatin;
Antimetabolites, exemplified by methotrexate (MTX), 6-thiopurines (mercaptopurine [6-mp], thioguanine [6-TG]), mercaptopurine (purinethol), thioguanine, fludarabine phosphate, cladribine: (leustatin), pentostatin, flurouracil (5-Fu), cytarabine (ara-C), and azacitidine;
Plant alkaloids, terpenoids and topoisomerase inhibitors, exemplified by vinblastine (velban), vincristine (oncovin), vindesine, vinorelbine, podophyllotoxins (etoposide (VP- 16) and teniposide (VM-26)), camptothecins (topotecan and irinotecan), taxanes such as paclitaxel (taxol) and docetaxel (taxotere);
Antibiotics, exemplified by doxorubicin (adriamycin, rubex, doxil), daunorubicin, idarubicin, dactinomycin (cosmegen), plicamycin (mithramycin), mitomycin: (mutamycin), and bleomycin (blenoxane);
Hormonal agents, exemplified by estrogen and androgen inhibitors (tamoxifen and flutamide), gonadotropin-releasing hormone agonists (leuprolide and goserelin (Zoladex)), and aromatase inhibitors (aminoglutethimide and anastrozole (arimidex));
Miscellaneous Anticancer Drugs, exemplified by amsacrine, asparaginase (El-spar), hydroxyurea, mitoxantrone (novantrone), mitotane (lysodren), retinoic acid derivatives, bone marrow growth factors (sargramostim and filgrastim), and amifostine;
Agents disrupting folate metabolism, e.g., pemetrexed;
DNA hypomethylating agents, e.g., azacitidine, and decitabine;
Poly(adenosine diphosphate [ADP]-ribose) polymerase (PARP) pathway inhibitors, such as iniparib, olaparib, and veliparib;
PI3K/Akt/mTOR pathway inhibitors, e.g., everolimus;
Histone deacetylase (HDAC) inhibitors, e.g., vorinostat, entinostat (SNDX-275), mocetinostat (MGCD0103), panobinostat (LBH589), romidepsin, valproic acid;
Cyclin-dependent kinase (CDK) inhibitors, e.g., flavopiridol, olomoucine, roscovitine, kenpaullone, AG-024322 (Pfizer), fascaplysin, ryuvidine, purvalanol A, NU2058, BML-259, SU 9516, PD-0332991, and P276-00;
Heat shock protein (HSP90) inhibitors, e.g., geldanamycin, tanespimycin, alvespimycin, radicicol, deguelin, and BIIB021;
Murine double minute 2 (MDM2) inhibitors, e.g., cis-imidazoline, benzodiazepinedione, spiro-oxindoles, isoquinolinone, thiophene, 5-deazaflavin, and tryptamine;
Anaplastic lymphoma kinase (ALK) inhibitors, e.g., aminopyridine, diaminopyrimidine, pyridoisoquinoline, pyrrolopyrazole, indolocarbazole, pyrrolopyrimidine, and dianilinopyrimidine;
Poly [ADPribose] polymerase (PARP) inhibitors, illustrated by benzamide, phthalazinone, tricyclic indole, benzimidazole, indazole, pyrrolocarbazole, phthalazinone, and isoindolinone;
A platinum-based drug, an antimetabolite, a BCL-2 inhibitor, a BTK inhibitor, a P13Kδ inhibitor, an anti-CD20 antibody, such as rituximab, obinutuzumab, ibritumomab tiuxetan, tositumomab, and veltuzumab, or a combination thereof; or
ABT-199 (Venetoclax), rituximab (RITUXAN®, MABTHERA®, ZYTUX®), ibrutinib (IMBRUVICA®), idelalisib (ZYDELIG®), tofacitinib, or a combination thereof.

In some embodiments, the other chemotherapeutic agent is a p90RSK inhibitor, such as those described in Cohen et al., “A clickable inhibitor reveals context-dependent autoactivation of p90 RSK,” Nat Chem Biol. 2007 Mar; 3(3): 156-160, and US Pat. No. 7605241. In one aspect, the p90RSK inhibitor is one or more of dexamethasone, melphalan, doxorubicin, bortezomib, lenalidomide, prednisone, carmustine, etoposide, cisplatin, vincristine, cyclophosphamide, BI-D1870, and thalidomide.

In some embodiments, a kinase inhibitor of the present disclosure (e.g., cerdulatinib) can be administered with a proton pump inhibitor, such as esomeprazole, omeprazole, lansoprazole, rabeprazole, dexlansoprazole, or a pharmaceutically acceptable salt thereof.

The specific amount of the agent co-administered with a kinase inhibitor of the present disclosure (e.g., cerdulatinib) described herein refers to the amount of the co-administered agent as a free base. However, it is understood that a pharmaceutically acceptable salt, co-crystal or solvate of the agent co-administered with a kinase inhibitor of the present disclosure (e.g., cerdulatinib) or a mixture thereof may be administered in an amount that provides the stated amount of the co-administered agent. Examples of pharmaceutically acceptable salts of the agent co-administered with a kinase inhibitor of the present disclosure (e.g., cerdulatinib) include those derived from inorganic or organic acids, such as acetate, adipate, alginate, aspartate, benzoate, benzene sulfonate, bisulfate, butyrate, citrate, camphorate, camphor sulfonate, cyclopentanepropionate, digluconate, dodecylsulfate, ethane sulfonate, edisylate, fumarate, lucoheptanoate, glycerophosphate, hemisulfate, heptanoate, hexanoate, hydrochloride, bis-hydrochloride, hydrobromide, hydroiodide, 2-hydroxyethanesulfonate, lactate, maleate, methanesulfonate, bis-methanesulfonate, 2-naphthalenesulfonate, naphthalene disulfate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulfates, phosphates, nitrates, sulphamates, malonates, salicylates, methylene-bis-b-hydroxynaphthoates, gentisates, isethionates, di-ptoluoyltartrates, ethanesulphonates, cyclohexylsulphamates, quinates, and the like.

The co-administered agent or a salt thereof can be administered in unsolvated forms as well as solvated forms, including hydrated forms, or form co-crystals with another compound. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate, etc. Some examples of solvents include, but are not limited to, methanol, N,N-dimethylformamide, tetrahydrofuran, dimethylsulfoxide, and water.

In some embodiments, the effective amount of the agent co-administered with a kinase inhibitor (e.g., cerdulatinib) of the present disclosure is at least about 5 mg per day. In some embodiments, the effective amount of the co-administered is at least about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg per day. In some embodiments, the effective amount of the co-administered agent is at least about 10, 20, 30, 40, or 50 mg per dosage. In some embodiments, the effective amount of the co-administered agent is at least about 15 mg, 20 mg, 25 mg, 30 mg, or 35 mg per dosage and is administered twice daily.

In some embodiments, the effective amount of the agent co-administered with a kinase inhibitor of the present disclosure (e.g., cerdulatinib) is no more than about 100 mg, 95 mg, 90 mg, 85 mg, 80 mg, or 75 mg of per day. In some embodiments, the effective amount of the co-administered agent is no more than about 90 mg, 80 mg, 70 mg, 60 mg, 55 mg or 50 mg per dosage. In some embodiments, the effective amount of the co-administered agent is no more than 45 mg, 40 mg, 35 mg, or 30 mg per dosage and is administered twice daily.

In some embodiments, the effective amount of the agent co-administered with a kinase inhibitor of the present disclosure (e.g., cerdulatinib) is from about 10 mg to 200 mg, from about 10 mg to 150 mg, from about 25 mg to 150 mg, from about 25 to 120 mg, from 30 mg to 110 mg, from about 50 to 120 mg, from about 30 to 80 mg, from 50 mg to 80 mg, from about 40 to 50 mg or from about 80 to 100 mg per day.

In some embodiments, the daily dose of the agent co-administered with a kinase inhibitor of the present disclosure (e.g., cerdulatinib) is about 30 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, or 150 mg.

In some embodiments, from about 30 mg to about 80 mg of the agent is co-administered with a kinase inhibitor of the present disclosure (e.g., cerdulatinib) once a day. In some embodiments, the effective amount is about 40 mg, 45 mg, 50 mg, 55 mg, 60 mg, 65 mg, or 70 mg of the co-administered agent once daily.

In some embodiments, the effective amount of the agent co-administered with a kinase inhibitor of the present disclosure (e.g., cerdulatinib) is about 15 mg to about 65 mg, about 25 mg to about 50 mg, about 25 mg to about 40 mg, about 30 mg to about 40 mg, or about 40 mg to about 50 mg per dosage administered twice daily. In some embodiments, about 25 mg, 30 mg, 35 mg, 40 mg, 45 mg, 50 mg, 55 mg, or 60 mg of the agent is co-administered twice daily. In some embodiments, about 45 mg of the agent is co-administered twice daily. In some embodiments, about 35 mg of the agent co-administered with twice daily.

In some embodiments, the effective amount of the agent co-administered with a kinase inhibitor of the present disclosure (e.g., cerdulatinib) is a daily dosage of from about 30 mg to about 90 mg. In some embodiments, the effective amount of the co-administered agent is a daily dosage of from about 40 mg, about 50 mg, about 60 mg, or about 70 mg, administered once or twice daily. In some embodiments, the effective amount of the co-administered agent is about 35 mg twice daily. In some embodiments, the effective amount of the co-administered agent is about 30 mg twice daily. In some embodiments, the effective amount of the co-administered agent is about 25 mg twice daily. In some embodiments, the effective amount of the co-administered agent is about 20 mg twice daily. In some embodiments, the effective amount of the co-administered agent is about 30 mg twice daily, and is reduced about 25 mg twice daily. In some embodiments, the effective amount of the co-administered agent is further reduced to about 20 mg twice daily. In some embodiments, the effective amount of the co-administered agent is further reduced to about 15 mg twice daily.

In some embodiments, the effective amount of the agent co-administered with a kinase inhibitor of the present disclosure (e.g., cerdulatinib) is between about 0.01 and 200 mg/kg. In some embodiments, about 0.01 and 150 mg/kg of agent may be co-administered. In other embodiments, a dosage of between 0.05 and 100 mg/kg of the agent may be co-administered. The dosage is described as a total amount of co-administered agent administered per time period. The dosage of the co-administered agent may be between about 0.1 mg and 2,000 mg/time period, between about 1 to 2,000 mg/time period, between about 1 to 1,000 mg/time period, between about 1 to 500 mg/time period, between about 10 to 150 mg/time period, between about 1 to 100 mg/time period, between about between about 1 to 50 mg/time period, between about 5 to 100 mg/time period, between about 10 to 125 mg/time period, between about 10 to 100 mg/time period, or between about 5 to 200 mg/time period. The dosage of the co-administered agent may be administered all in one time (once per time period) or in several times, such as two times, three times, four times, five times or more throughout the time period. In some embodiments, the time period is, or is about, every week, every two weeks, every three weeks, every four weeks, every one month, every two months, every three months, every four months, every five months, every six months, every seven months, every eight months, every nine months, every ten months, every eleven months, every year, or a number or a number or a range between any two of these values.

In some embodiments, the co-administered agent (e.g., rituximab) is administered in the dosage forms and strengths of 100 mg/10 mL (10 mg/mL) and 500 mg/50 mL (10 mg/mL) solution, for example, in single-dose vials.

In some embodiments, the effective amount of the co-administered agent is an amount determined by a medical practitioner for treatment of the disease or indication.

In some embodiments, the effective amount of the co-administered agent (e.g., for the dose of the first cycle and for the doses of the subsequent cycles) is, or is about, 100 mg/m², 110 mg/m², 120 mg/m², 130 mg/m², 140 mg/m², 150 mg/m², 160 mg/m², 170 mg/m², 180 mg/m², 190 mg/m², 200 mg/m², 210 mg/m², 220 mg/m², 230 mg/m², 240 mg/m², 250 mg/m², 260 mg/m², 270 mg/m², 280 mg/m², 290 mg/m², 300 mg/m², 310 mg/m², 320 mg/m², 330 mg/m², 340 mg/m², 350 mg/m², 360 mg/m², 370 mg/m², 375 mg/m², 380 mg/m², 390 mg/m², 400 mg/m², 410 mg/m², 420 mg/m², 430 mg/m², 440 mg/m², 450 mg/m², 460 mg/m², 470 mg/m², 480 mg/m², 490 mg/m², 500 mg/m², 510 mg/m², 520 mg/m², 530 mg/m², 540 mg/m², 550 mg/m², 560 mg/m², 570 mg/m², 580 mg/m², 590 mg/m², 600 mg/m², 610 mg/m², 620 mg/m², 630 mg/m², 640 mg/m², 650 mg/m², 660 mg/m², 670 mg/m², 680 mg/m², 690 mg/m², 700 mg/m², 710 mg/m², 720 mg/m², 730 mg/m², 740 mg/m², 750 mg/m², 760 mg/m², 770 mg/m², 780 mg/m², 790 mg/m², 800 mg/m², 810 mg/m², 820 mg/m², 830 mg/m², 840 mg/m², 850 mg/m², 860 mg/m², 870 mg/m², 880 mg/m², 890 mg/m², 900 mg/m², 910 mg/m², 920 mg/m², 930 mg/m², 940 mg/m², 950 mg/m², 960 mg/m², 970 mg/m², 980 mg/m², 990 mg/m², 1000 mg/m², or a number or a number or a range between any two of these values, per dose or time period for a number of times or cycles.

In some embodiments, the effective amount of the co-administered agent per dose is from about 50 mg to about 1000 mg every three to five weeks up to five to seven times. In some embodiments, the effective amount of the co-administered agent is from about 100 mg to about 500 mg every three to five weeks up to six times. In some embodiments, the effective amount of the co-administered agent is from about 100 mg to about 500 mg every 28 days up to six times. In some embodiments, the effective amount of the co-administered agent (e.g., for the dose of the first cycle and for the doses of the subsequent cycles) is, or is about, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg 1000 mg, or a number or a number or a range between any two of these values, per dose or time period for a number of times or cycles.

In some embodiments, the effective amount of the co-administered agent is once weekly for about 4 or 8 doses. In some embodiments, the effective amount of the co-administered agent is once weekly for about 4 doses. In some embodiments, the time period between two consecutive doses is, or is about, every day, every two days, every three days, every four days, every five days, every six days, every week, every two weeks, every three weeks, every four weeks, every one month, every two months, every three months, every four months, every five months, every six months, every seven months, every eight months, every nine months, every ten months, every eleven months, every twelve months, or a number or a number or a range between any two of these values. In some embodiments, the number of times or cycles is, is about, or is at most, 2, 3, 4, 5, 6, 7, 8, 9, 10, 11, 12, 13, 14, 15, 16, 17, 18, 19, 20, or a number or a number or a range between any two of these values.

In some embodiments, the dose for the first cycle and the doses of the subsequent cycles are identical. In some embodiments, the dose for the first cycle and the doses of the subsequent cycles are different.

In some embodiments, the effective amount of the co-administered agent includes two initial doses separated by about two weeks and subsequent doses every six months thereafter. In some embodiments, the effective amount of the co-administered agent includes two initial doses separated by about two weeks and subsequent doses at month 12 and every six months thereafter. In some embodiments, the effective amount of the co-administered agent includes a number of initial doses, such as 1 dose, 2 doses, 3 doses, 4 dose, 5 doses, 6 doses, 7 doses, 8 doses, 9 doses, or 10 doses. In some embodiments, the two consecutive initial doses are separated by, or by about, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, or a number or a number or a range between any two of these values. In some embodiments, subsequent doses are administered starting, or starting about, 6 months, 7 months, 8 months, 9 months, 10 months, 11 months, 12 months, 13 months, 14 months, 15 months, 16 months, 17 months, 18 months, or a number or a number or a range between any two of these values. In some embodiments, the subsequent doses are administered, or administered about, every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, or a number or a number or a range between any two of these values. In some embodiments, the number of subsequent doses is, or is about, 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 19 doses, 19 doses, 20 doses, or a number or a number or a range between of these two values.

In some embodiments, the co-administered agent (e.g., in combination with a kinase inhibitor of the present disclosure (e.g., cerdulatinib)) is used for maintenance after complete or partial response begins about eight weeks following completion of a the co-administered agent product. In some embodiments, the co-administered agent used for maintenance is administered every 8 weeks for 12 doses. In some embodiments, the co-administered agent is used for maintenance after complete or partial response following, or following about, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or a number or a number or a range between of these two values, completion of a the co-administered agent product. In some embodiments, the co-administered agent used for maintenance is administered, or administered about, every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or a number or a number or a range between of these two values. In some embodiments, the co-administered agent used for maintenance is administered, or administered about, 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 19 doses, 19 doses, 20 doses, or a number or a number or a range between of these two values.

In some embodiments, the effective amount of the co-administered agent is co-administered with a kinase inhibitor of the present disclosure (e.g., cerdulatinib) at from about 100 mg/10 mL to about 500 mg/50 mL. In some embodiments, the effective amount of the co-administered agent is co-administered with a kinase inhibitor of the present disclosure (e.g., cerdulatinib) at from about 100 mg/10 mL to about 200 mg/20 mL. In some embodiments, the effective amount of the co-administered agent is co-administered with a kinase inhibitor of the present disclosure (e.g., cerdulatinib) at from about 200 mg/20 mL to about 300 mg/30 mL. In some embodiments, the effective amount of the co-administered agent is co-administered with a kinase inhibitor of the present disclosure (e.g., cerdulatinib) at from about 300 mg/30 mL to about 400 mg/40 mL. In some embodiments, the effective amount of the co-administered agent is co-administered with a kinase inhibitor of the present disclosure (e.g., cerdulatinib) at from about 400 mg/40 mL to about 500 mg/50 mL.

In some embodiments, the effective amount of the co-administered agent is administered in a concentration of about 5 mg/mL. In some embodiments, the effective amount of the co-administered agent is administered in a concentration of about 10 mg/mL. In some embodiments, the effective amount of the co-administered agent is administered in a concentration of about 15 mg/mL. In some embodiments, the effective amount of the co-administered agent is administered in a concentration of from about 5 mg/mL to about 15 mg/mL. In some embodiments, the effective amount of the co-administered agent is administered in a concentration of from about 5 mg/mL to about 10 mg/mL. In some embodiments, the effective amount of the co-administered agent is administered in a concentration of from about 10 mg/mL to about 15 mg/mL. In some embodiments, the effective amount of the co-administered agent administered in a concentration of from about 15 mg/mL to about 20 mg/mL. In some embodiments, the effective amount of the co-administered agent is administered in a concentration of about 1 mg/mL, 2 mg/mL, 3 mg/mL, 4 mg/mL, 5 mg/mL, 6 mg/mL, 7 mg/mL, 8 mg/mL, 9 mg/mL, 10 mg/mL. 11 mg/mL, 12 mg/mL, 13 mg/mL, 14 mg/mL, 15 mg/mL, 16 mg/mL, 17 mg/mL, 18 mg/mL, 19 mg/mL, 20 mg/mL. 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, or a number or a range between any two of these values.

In some embodiments, provided is a composition comprising a kinase inhibitor of the present disclosure (e.g., cerdulatinib) and a co-administered agent (e.g., rituximab). In some embodiments, the molar ratio of the kinase inhibitor to the co-administered agent is about 300:1 to about 3:1. In some embodiments, the composition comprises the kinase inhibitor to the co-administered agent in a molar ratio of about 9:1 to about 1:9. In some embodiments, the composition comprises the kinase inhibitor to the co-administered agent in a molar ratio of about 2:1 to about 1:2. In some embodiments, the composition comprises the kinase inhibitor to the co-administered agent in a molar ratio of about 2:1 to about 1:5. In some embodiments, the composition comprises the kinase inhibitor to the co-administered agent in a molar ratio of about 1:1. In some embodiments, the composition comprises the kinase inhibitor to the co-administered agent in a molar ratio of about 1:1, about 1:2, about1:9, about 2:1, or about 9:1. In some embodiments, the composition comprises the kinase inhibitor to the co-administered agent in a molar ratio of, of about, of at least, or of at most, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or a number or a number or a range between any two of these values. In some embodiments, the composition comprises the kinase inhibitor to the co-administered agent in a molar ratio of, of about, of at least, or of at most, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, or a number or a number or a range between any two of these values.

### 6. Pharmaceutical Compositions and Kits

Some embodiments provided herein are directed to pharmaceutical compositions comprising an effective amount of the kinase inhibitor (e.g., cerdulatinib) and at least one pharmaceutically acceptable carrier or excipient.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” and the term “pharmaceutically acceptable excipient” refer to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the cancerous tissue or a tissue adjacent to the cancerous tissue.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds disclosed herein are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “formulated” or “formulation” refers to the process in which different chemical substances, including one or more pharmaceutically active ingredients, are combined to produce a dosage form. In certain embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

Carriers or excipients can be used to produce compositions. The carriers or excipients can be chosen to facilitate administration of the kinase inhibitor (e.g., cerdulatinib). Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in The Science and Practice of Pharmacy, 21st edition, Lippincott, Williams and Wilkins, Philadelphia, Pa., 2005 (hereby incorporated by reference herein).

The kinase inhibitor (e.g., cerdulatinib) can be administered by different routes including intravenous, intraperitoneal, subcutaneous, intramuscular, oral, transmucosal, rectal, transdermal, or inhalant. In some embodiments, the kinase inhibitor (e.g., cerdulatinib) can be administered by oral administration. For oral administration, for example, the kinase inhibitor (e.g., cerdulatinib) can be formulated into conventional oral dosage forms such as capsules, tablets, and liquid preparations such as syrups, elixirs, and concentrated drops.

For inhalants, the kinase inhibitor (e.g., cerdulatinib) may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The kinase inhibitor (e.g., cerdulatinib) may also be used in combination with other inhaled therapies, for example corticosteroids such as fluticasone propionate, beclomethasone dipropionate, triamcinolone acetonide, budesonide, and mometasone furoate; beta agonists such as albuterol, salmeterol, and formoterol; anticholinergic agents such as ipratropium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

Pharmaceutical preparations for oral use can be obtained, for example, by combining the kinase inhibitor (e.g., cerdulatinib) with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP, povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the kinase inhibitor (e.g., cerdulatinib) may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the kinase inhibitor (e.g., cerdulatinib) can be formulated in sterile liquid solutions, such as in physiologically compatible buffers or solutions, such as saline solution, Hank’s solution, or Ringer’s solution. In addition, the kinase inhibitor (e.g., cerdulatinib) may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

In some embodiments, the present disclosure provides kits that include the kinase inhibitor (e.g., cerdulatinib, or a pharmaceutically acceptable salt thereof) or a pharmaceutical composition thereof. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a protein kinase mediated disease or condition; the kits described herein may include written instructions for use and/or other indication that the compound or composition is suitable or approved for administration to a mammal, e.g., a human, for a disease or condition as described herein, such as a hematologic cancer; and the compound or composition may be packaged in unit dose or single dose form, e.g., single dose pills, capsules, or the like.

### EXAMPLES

Examples related to the present disclosure are described below. In certain cases, alternative techniques can be used. The examples are intended to be illustrative and are not limiting or restrictive to the scope of the disclosure.

### Example 1

### Identifying Patient Groups With Differential Clinical Features and Response to Cerdulatinib

This example describes a phase IIa study designed to determine the safety, efficacy, pharmacokinetics, and pharmacodynamics of cerdulatinib at the selected phase II dose of 30 mg BID, dosed in 28 day cycles, in patients with r/r B or T cell malignancies.

The dual spleen tyrosine kinase (SYK) and Janus kinase (JAK) inhibitor cerdulatinib was recently evaluated for safety and efficacy in patients with relapsed/refractory (r/r) B and T cell malignancies. SYK is required for B cell antigen receptor signaling, facilitating B cell development at all stages beyond pro-B cells. Cytokines via JAK signaling similarly direct B cell development. Hence, as part of the phase IIa dose expansion study in patients with indolent B cell lymphoma (iNHL), the effect of cerdulatinib on B cell subsets over time was assessed. Age-matched healthy controls were also evaluated to enable a comparison of B cell subsets between healthy and disease, as skewing of B cell subsets was previously reported in iNHL.

Blood was collected from 38 patients at baseline, 2, 3, and 6 months following initiation of therapy for the evaluation of treatment-related effects on circulating B cell subsets. The initial hypothesis was that as a consequence of prolonged SYK and JAK inhibition an impact on B cell subsets would be observed over time. One goal of the study of this example was to determine if any particular subpopulation was sensitive to the effects of cerdulatinib. Aside from changes to some cell surface markers, very little impact to the measured B cell populations was observed over a period of 6 months on therapy. Rather, considerable differences between healthy and iNHL B cell subpopulations at baseline was found, which had not been previously reported.

The diversity in B cell subset skewing at baseline in the lymphoma patients was evaluated using an unsupervised multivariate analysis. Hierarchical clustering revealed 3 distinct patient groups. Group differences were driven primarily by diversity in the frequencies of transitional, naïve, switched memory, and double negative B cell subsets. Importantly, these 3 phenotypically distinct groups were found to have different clinical features and different responses to cerdulatinib treatment.

Relative to healthy, Group 1 patients at baseline exhibited high naïve B cell content, of which there was observed an overrepresentation of CD10+ transitional B cells. Group 2 patients at baseline also exhibited high naïve B cell content, but with normal frequency of transitional B cells. Group 3 patients at baseline exhibited low naïve B cell content relative to healthy and high frequency of switched memory (skewed toward IgM over IgG) or double negative B cells. The overall response rates for Groups 1, 2, and 3 were 22%, 81%, and 46%, respectively; the median time on drug (in weeks) for these groups was 8, 59, and 16, respectively. Of note, 78% of Group 1 patients discontinued therapy due to adverse event, compared to 13% and 23% for Groups 2 and 3, respectively. These data suggest that iNHL circulating B cell phenotype assessment may predict clinical response, both safety and efficacy, to cerdulatinib, an observation which will be confirmed in a follow-on clinical trial.

### Materials and Methods

### Phase IIa Study

Up to 50 patients were enrolled in 5 cohorts; aggressive lymphoma, iNHL, CLL/SLL, PTCL (all receiving single agent cerdulatinib), and iNHL with cerdulatinib in combination with anti-CD20 therapy. Patients remained on drug until discontinuation due to progressive disease (PD), adverse event (AE), withdrawal of consent (WC), or at the discretion of the treating physician.

Forty patients with iNHL were enrolled in a cohort receiving single agent cerdulatinib. From this cohort, 30 ml of whole blood was collected from each patient at pre-dose at baseline (C1), cycle 2 day 1 (C2), cycle 3 day 1 (C3), and cycle 6 day 1 (C6) in 10 ml mononuclear cell protection tubes (CPT tubes, BD Bioscience, San Jose, CA) and immediately centrifuged to separate mononuclear cells from plasma and red blood cells at each clinical site. At each time point, tubes were shipped for same day delivery to Caprion Biosciences, Montreal Canada, for preparation and live cell freezing of peripheral blood mononuclear cells (PBMC) by ficoll gradient centrifugation using standard procedures. Banked PBMC’s were stored at -80° C. until 5-8 patient samples through cycle 6 were obtained to enable batched flow cytometry analysis of B cell subsets for each patient over time.

### B Cell Subpopulation Analysis

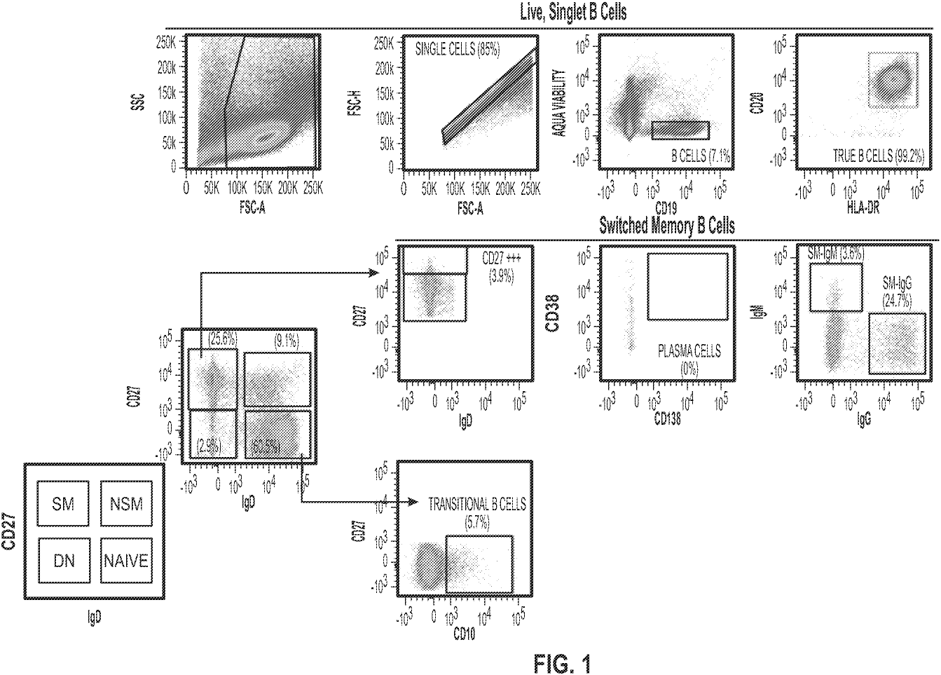

PBMC’s were thawed and rested overnight in RPMI containing 10% fetal bovine serum. Cells were then stained with a cocktail containing Aqua live/dead cell stain (Life Technologies, Carlsbad, CA), and the following antibodies all obtained from BD Biosciences; CD19 PE-Cy7, CD20 BUV395, HLA-DR APC-Cy7, CD27 BV786, IgD PE, CD10 BV650, CD38 BV711, CD138 BV421, IgG BV605, and IgM APC. Cells were stained for 1 hour at room temperature, washed in phosphate buffered saline containing 0.5% bovine serum albumin, and subjected to flow cytometry using the BD Cantos. A maximum number of events was collected, the median number of total B cells collected was 18,789. The B cell subset gating strategy is described in FIG. 1. As shown in FIG. 1, cell debri was gated out, followed by gating of single cells by forward scatter area by forward scatter height.

Live B cells were then gated based on aqua viability stain and CD19. IgD and CD27 delineated double negative (DN), naïve, switched memory (SM), and non-switched memory (NSM). Within the naive cell area transitional B cells were identified based on CD10 expression. Within the SM area IgG and IgM switched B cells were identified. Plasma cells were identified based on high CD27 expression and CD138. Percent lymphocytes of total live PBMC’s were also calculated. All gating and calculation of B cell subset frequencies either from the total B cell gate or the immediate parent gate of a B cell subset were calculated using Flow Jo software (Tree Star, Inc., Ashland, OR), and analyzed. Table 1 shows details of the antibodies used for the B cell flow cytometry analysis.

TABLE 1

Antibodies used in B cell flow cytometry analysis

| Marker | Identify | Fluorochrome | Clone | Supplier | Catalog # | Expression |
|---|---|---|---|---|---|---|
| Live/Dead | Live Cells | Aqua | N/Ap | Life Technologies | L34957 | Constitutive |
| CD19 | B Cells | PE-Cy7 | SJ25-C1 | BD | 557835 | Constitutive |
| CD20 | B Cells | BUV395 | 2H7 | BD | 563782 | Constitutive |
| HLA-DR | B Cells | APC-Cy7 | L243 | BD | 335796 | Constitutive |
| CD27 | Switch/Memory | BV786 | L128 | BD | 563327 | Constitutive |
| IgD | Naïve | PE | IA6-2 | BD | 555779 | Constitutive |
| CD 10 | Naive/Transition | BV650 | HI10a | BD | 563734 | Constitutive |
| CD38 | Ab Secretion | BV711 | HIT2 | BD | 563965 | Constitutive |
| CD138 | Ab Secretion | BV421 | MI15 | BD | 562935 | Constitutive |
| IgG | Switch | BV605 | G18-145 | BD | 563246 | Constitutive |
| IgM | Switch | APC | G20-127 | BD | 551062 | Constitutive |

### Results

### Principle Component Analysis of iNHL B Cell Subsets

PBMC’s serially collected from 38 iNHL patients treated with single agent cerdulatinib were evaluated for frequencies of various B cell subsets. Baseline samples were collected from all patients, and from 84%, 84%, and 50% of patients at cycles 2, 3, and 6 post therapy initiation, respectively. For comparison, 6 age-matched healthy subject control PBMC’s were also assessed using the same methods.

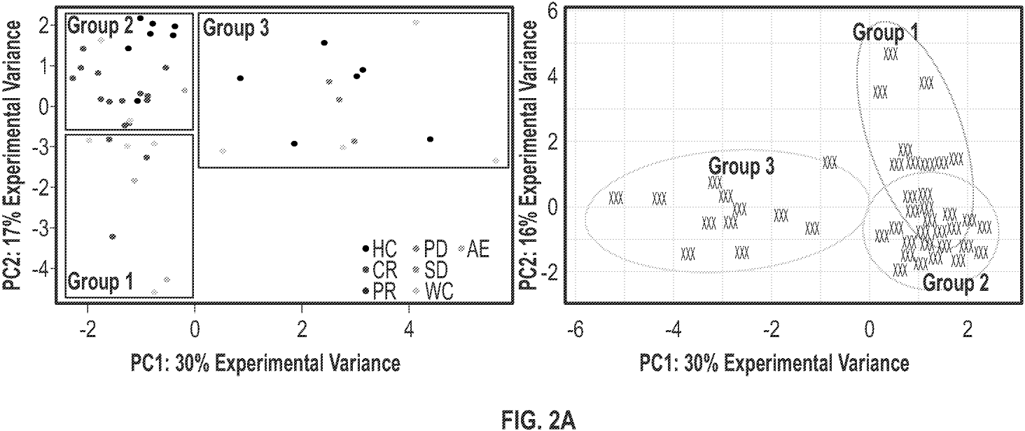

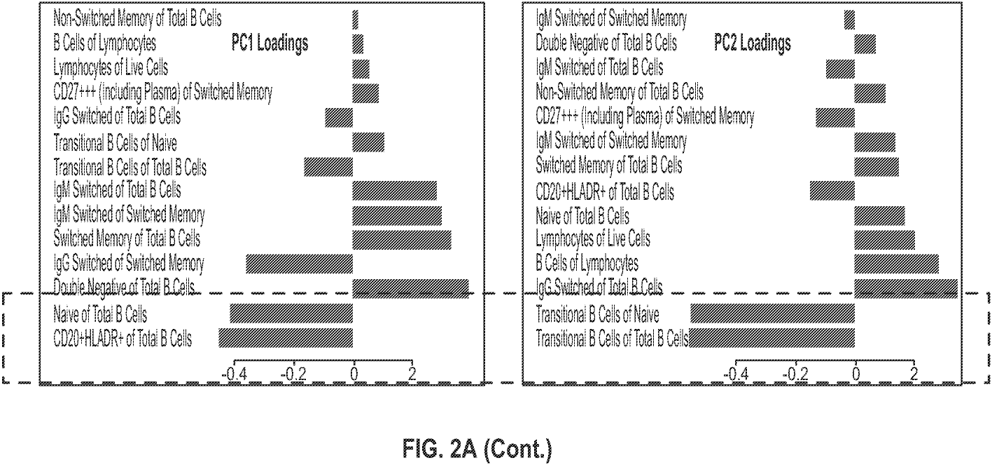

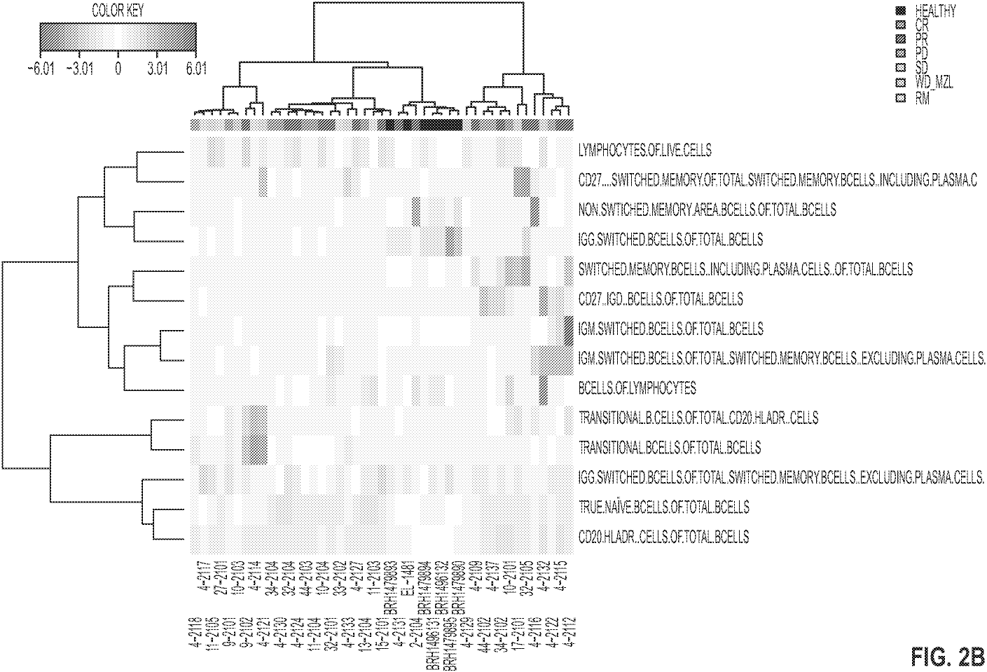

Representative gating for the B cell subsets evaluated is shown in FIG. 1. At baseline, significant skewing of B cell subset frequencies was observed in iNHL patients relative to healthy. A PCA analysis of the data was performed to identify subgroups of patients characterized by a common B cell phenotype. Subsequent analysis of the results of the PCA by hierarchical clustering was performed. Results of the PCA analysis are presented in FIG. 2A (top left). This unsupervised analysis of B cell subset frequencies revealed three distinct patient populations (referred to as Groups 1, 2, and 3), the hierarchical clustering of which is shown as a heat map in FIG. 2B. As shown in the heat map of FIG. 2B, the 6 healthy donors clustered to a common region, whereas the iNHL patients showed considerable diversity.

The main drivers of phenotypic diversity are shown in the PCA-loading plots in FIG. 2A (bottom left and right). Principle component 1 was driven primarily by skewing of naive, double negative, and switched memory B cells, whereas principle component 2 was driven primarily by skewing of transitional, IgG switched, naive, B cell and lymphocyte frequencies. The identified subgroups were confirmed by partial least squares (PLS) analysis, which demonstrated that these three phenotypically distinct patient groups can be separated with reasonable confidence. Shown in the top right of FIG. 2A is the result of this analysis, with groups identified along with 95% confidence ellipses. Group 3 did not overlap with Groups 1 and 2, whereas the latter two groups demonstrated overlap in confidence ellipses.

### Distinction of R/r iNHL Patient Subgroups by Clinical Features

Clinical features and response to cerdulatinib among the three above-identified and phenotypically defined groups were assessed. Patient demographics are shown in Table 2. Group 3 presented with the greatest median number of prior therapies (5, ranging from 1-7) and highest frequency of refractory disease (42%). Unique to patient Group 1 was the frequency of pre-diabetic/diabetic patients (56%).

TABLE 2

Baseline patient characteristics

| | G1 (N=9) | G2 (N=16) | G3 (N=13) | Overall (N=38) |
|---|---|---|---|---|
| Sex | | | | |
| F | 3.00 (33.3%) | 9.00 (56.3%) | 2.00 (15.4%) | 14.0 (36.8%) |
| M | 6.00 (66.7%) | 7.00 (43.8%) | 11.0 (84.6%) | 24.0 (63.2%) |
| Age | | | | |
| Mean (SD) | 64.3 (13.7) | 61.3 (8.87) | 64.0 (11.0) | 62.9 (10.7) |
| Median [Min, Max] | 64.0 [42.0, 81.0] | 61.5 [45.0, 76.0] | 65.0 [42.0, 81.0] | 64.0 [42.0, 81.0] |
| Time Since Initial Diagnosis (month) | | | | |
| Mean (SD) | 62.8 (57.3) | 103 (124) | 77.5 (66.4) | 84.9 (92.8) |
| Median [Min, Max] | 32.4 [14.2, 169] | 67.2 [0.200, 497] | 71.9 [3.00, 244] | 64.6 [0.200, 497] |

Count_regimen

| | | | | |
|---|---|---|---|---|
| Mean (SD) | 1.78 (1.09) | 2.38 (1.50) | 3.23 (2.05) | 2.53 (1.69) |
| Median [Min, Max] | 1.00 [1.00, 4.00] | 2.00 [1.00, 7.00] | 3.00 [1.00, 7.00] | 2.00 [1.00, 7.00] |

Rituxan

| Yes | 9.00 (100%) | 16.0 (100%) | 13.0 (100%) | 38.0 (100%) |
|---|---|---|---|---|
| Bendamustine | | | | |
| No | 6.00 (66.7%) | 13.0 (81.3%) | 13.0 (100%) | 32.0 (84.2%) |
| Yes | 3.00 (33.3%) | 3.00 (18.8%) | 0 (0%) | 6.00 (15.8%) |
| Anthracycline | | | | |
| No | 6.00 (66.7%) | 10.0 (62.5%) | 6.00 (46.2%) | 22.0 (57.9%) |
| Yes | 3.00 (33.3%) | 6.00 (37.5%) | 7.00 (53.8%) | 16.0 (42.1%) |
| Time_since_prior therapy | | | | |
| Mean (SD) | 680 (837) | 662 (412) | 346 (342) | 558 (540) |
| Median [Min, Max] | 441 [60.0, 2830] | 617 [56.0, 1360] | 225 [28.0, 924] | 441 [28.0, 2830] |
| Missing | 0 (0%) | 2.00 (12.5%) | 1.00 (7.7%) | 3.00 (7.9%) |

Consistently, as shown in Table 3, Group 1 patients exhibited the least median time on cerdulatinib at 8 weeks, compared to 59 weeks and 16 weeks for Groups 2 and 3, respectively. Group 1 patients have tolerated cerdulatinib the least, with 7 of 9 patients discontinuing study due to AE. Group 2 patients have experienced the greatest benefit to cerdulatinib, with not only a median of 59 weeks on therapy, but resulting in 75% of patients staying on therapy for greater than 180 days.

TABLE 3

Patient disposition

| | G1 (N=9) | G2 (N=16) | G3 (N=13) | G2 + G3 (N=29) | Total (N=38) |
|---|---|---|---|---|---|
| Discontinuation | | | | | |
| AE | 4.00 (44.4%) | 1.00 (6.3%) | 2.00 (15.4%) | 3.00 (10.3%) | 7.00 (18.4%) |
| Ongoing | 4.00 (44.4%) | 4.00 (25.0%) | 5.00 (38.5%) | 9.00 (31.0%) | 13.0 (34.2%) |
| PD | 1.00 (11.1%) | 6.00 (37.5%) | 5.00 (38.5%) | 11.0 (37.9%) | 12.0 (31.6%) |
| Other | 0 (0%) | 5.00 (31.3%) | 1.00 (7.7%) | 6.00 (20.7%) | 6.00 (15.8%) |
| Total Treatment Duration (weeks) | | | | | |
| Mean (SD) | 20.6 (30.4) | 68.2 (52.8) | 32.1 (41.6) | 52.0 (50.7) | 44.6 (48.2) |
| Median [Min, Max] | 8.00 [2.43, 92.3] | 59.1 [0.429, 174] | 16.0 [5.43, 159] | 35.7 [0.429, 174] | 26.1 [0.429, 174] |

Days on trial > 180

| | | | | | |
|---|---|---|---|---|---|
| No | 7.00 (77.8%) | 4.00 (25.0%) | 8.00 (61.5%) | 12.0 (41.4%) | 19.0 (50.0%) |
| Yes | 2.00 (22.2%) | 12.0 (75.0%) | 5.00 (38.5%) | 17.0 (58.6%) | 19.0 (50.0%) |

Abbreviations: PD, progressive disease; AE, adverse event

This is reflected in the clinical response rates, shown in Table 4. Group 1 had the lowest response rate at 22%, all PR’s. Group 2 had the greatest response rate at 81%, with a mixture of CR (25%) and PR (56%). Group 3 had a reasonable, albeit lower response rate than Group 2, with an ORR of 46%, exhibiting 17% CR and 38% PR.

TABLE 4

Clinical response summary

| | G1 (N=9) | G2 (N=16) | G3 (N=13) | G2 + G3 (N=29) | Total (N=38) |
|---|---|---|---|---|---|
| Best Response | | | | | |
| CR | 1.00 (11.1%) | 5.00 (31.3%) | 1.00 (7.7%) | 6.00 (20.7%) | 7.00 (18.4%) |
| PR | 1.00 (11.1%) | 8.00 (50.0%) | 6.00 (46.2%) | 14.0 (48.3%) | 15.0 (39.5%) |
| PD | 1.00 (11.1%) | 0 (0%) | 3.00 (23.1%) | 3.00 (10.3%) | 4.00 (10.5%) |
| ORR | 2.00 (22.2%) | 13.0 (81.3%) | 7.00 (53.8%) | 20.0 (69.0%) | 22.0 (57.9%) |
| SD | 2.00 (22.2%) | 1.00 (6.3%) | 3.00 (23.1%) | 4.00 (13.8%) | 6.00 (15.8%) |
| WD_MZL | 1.00 (11.1%) | 0 (0%) | 0 (0%) | 0 (0%) | 1.00 (2.6%) |
| RM | 3.00 (33.3%) | 2.00 (12.5%) | 0 (0%) | 2.00 (6.9%) | 5.00 (13.2%) |
| ORR + clinical benefit | 4.00 (44.4%) | 14.0 (87.5%) | 10.0 (76.9%) | 24.0 (82.8%) | 28.0 (73.7%) |

Abbreviations ORR, overall response rate; CR, complete response; PR, partial response;
SD, Stable disease; PD, progressive disease; WD MZL, Waldenstrom’s (WD) and marginal zone lymphoma (MZL)

### Elevations in CD10+ Transitional B Cells Is a Distinguishing Feature of Group 1 Patients

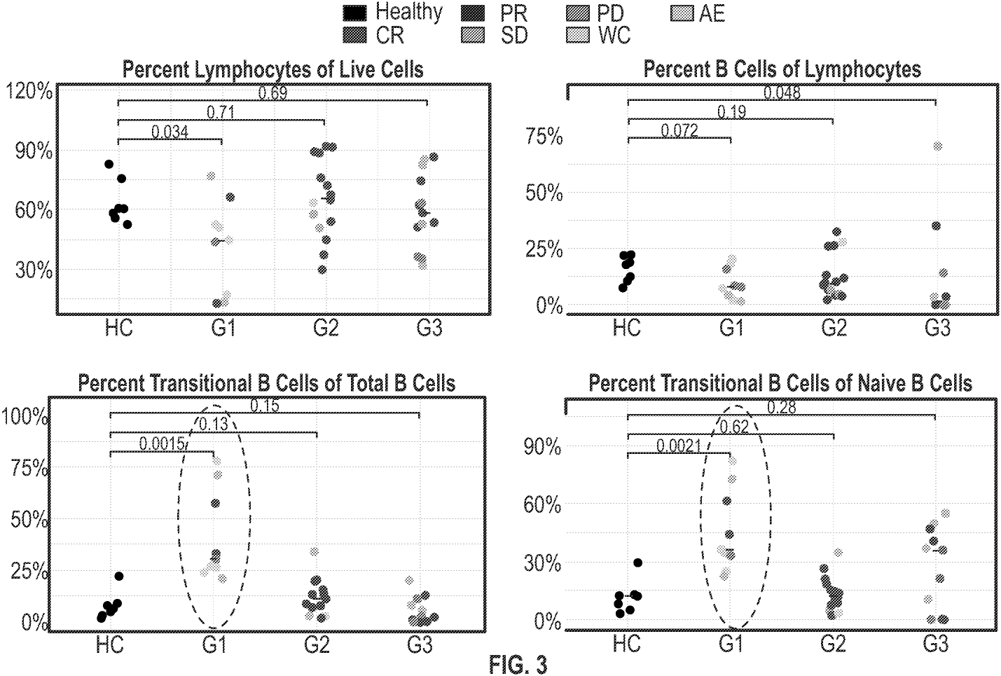

The PCA analysis showed that a main phenotypic difference identifying Group 1 patients was overrepresentation of transitional B cells of naïve and total B cells. The raw frequency data demonstrating this phenomenon is presented in FIG. 3. Whereas lymphocyte frequency of live cells (top left) and B cell frequency of lymphocytes (top right) were not notably different among healthy and iNHL groups, the transitional B cells of total B cells (bottom left) and of naïve B cells (bottom right) were significantly elevated in Group 1.

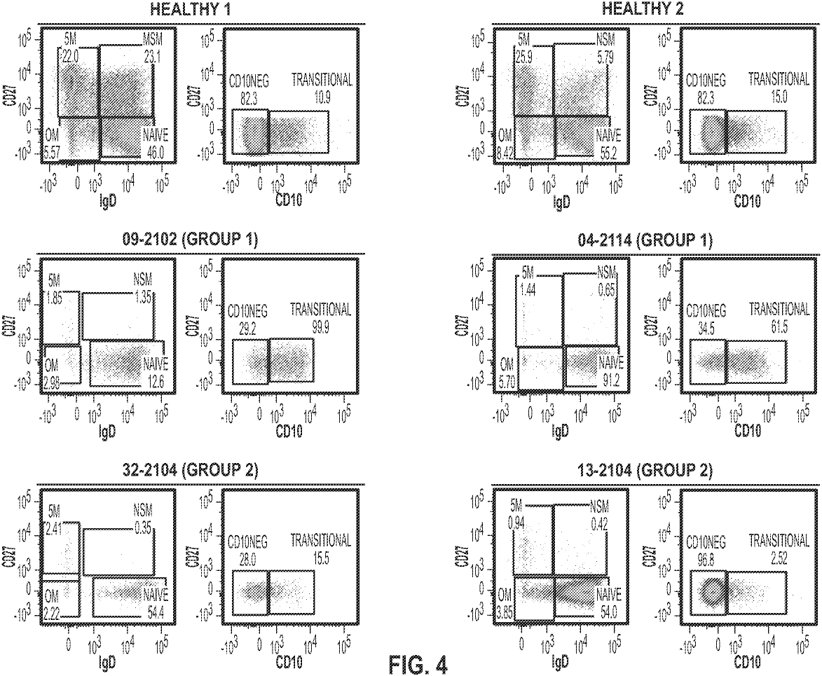

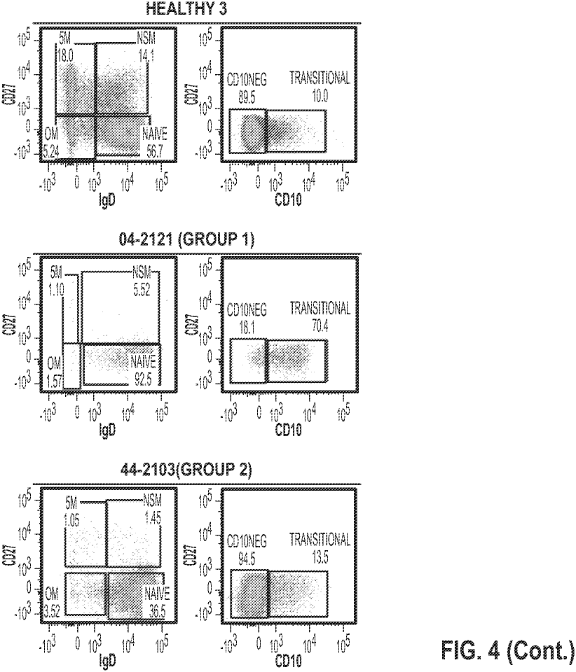

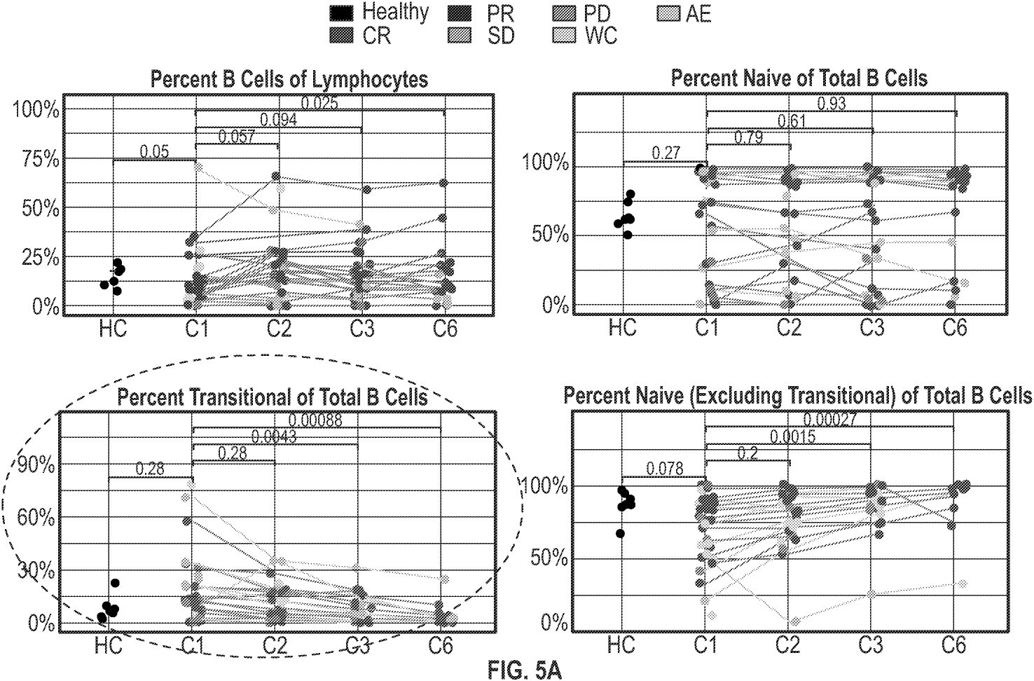

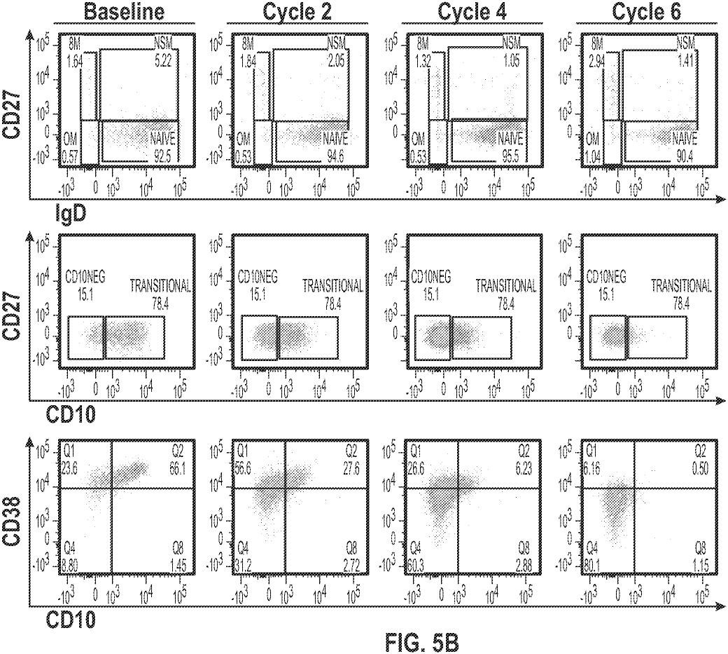

As shown in FIG. 2B, Groups 1 and 2 both had elevated naïve B cell populations relative to healthy, however, as seen in the raw FACS plots in FIG. 4, Group 1 iNHL patients uniquely present with higher frequency of CD10+ transitional B cells (middle row), relative to healthy (top row) and Group 2 patients (bottom row). The effect of cerdulatinib on B cell subset frequencies was plotted over time for all subsets evaluated (data not shown), and the only population with an apparent treatment-related change was loss of transitional B cells. Shown in FIG. 5A, while the frequency of B cells of lymphocytes (top left) and frequency of total naïve B cells (top right) did not change over time, the frequency of transitional B cells decreased (bottom left) with a rise in the frequency of CD10 negative naïve B cells (bottom right). These data indicated a loss in CD10 expression rather than a loss in the transitional B cell population. This is further demonstrated in FIG. 5B with a representative Group 1 patient with high baseline CD10 positive transitional B cells. As shown, the total naïve B cell population (top row) did not change in frequency with time on cerdulatinib, whereas the CD 10 positive population decreased over time resulting in a corresponding increase in “true, non-transitional” naive B cells (middle row). The cells was plotted as CD10 by CD38, both markers for transitional B cells, and observed that both CD10 and CD38 expression appear to be lost over time (bottom row). The data indicated that transitional B cells are not being lost over time with cerdulatinib treatment, but there is an observed loss of CD10 and CD38 expression.

### Elevations in Switched Memory or Double Negative B Cells With Corresponding Loss of Naïve Cells Is a Main Feature of Group 3 iNHL Patients

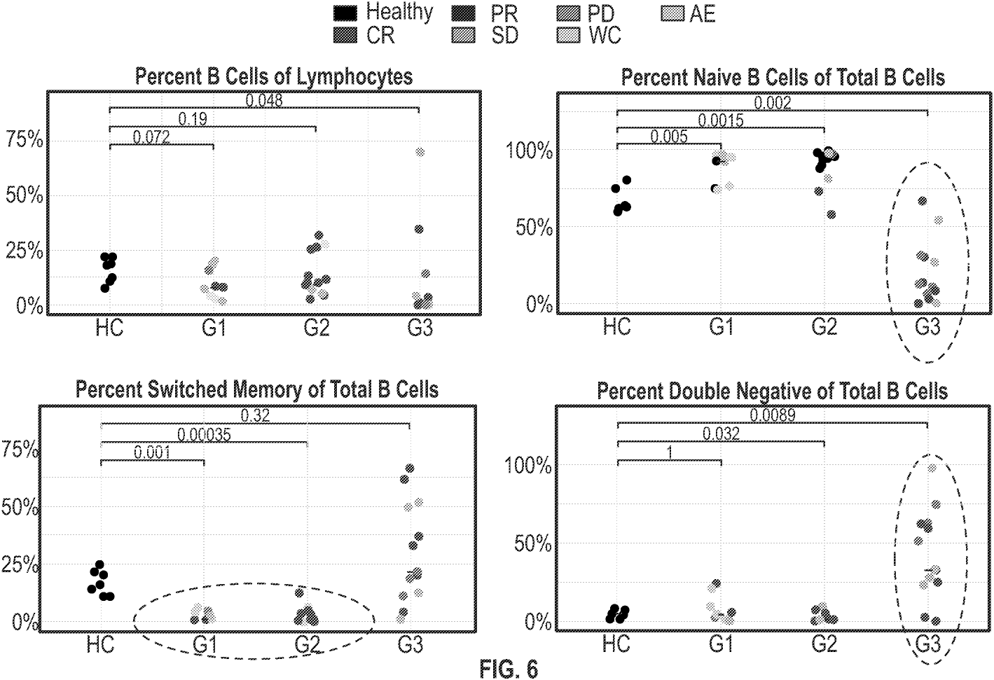

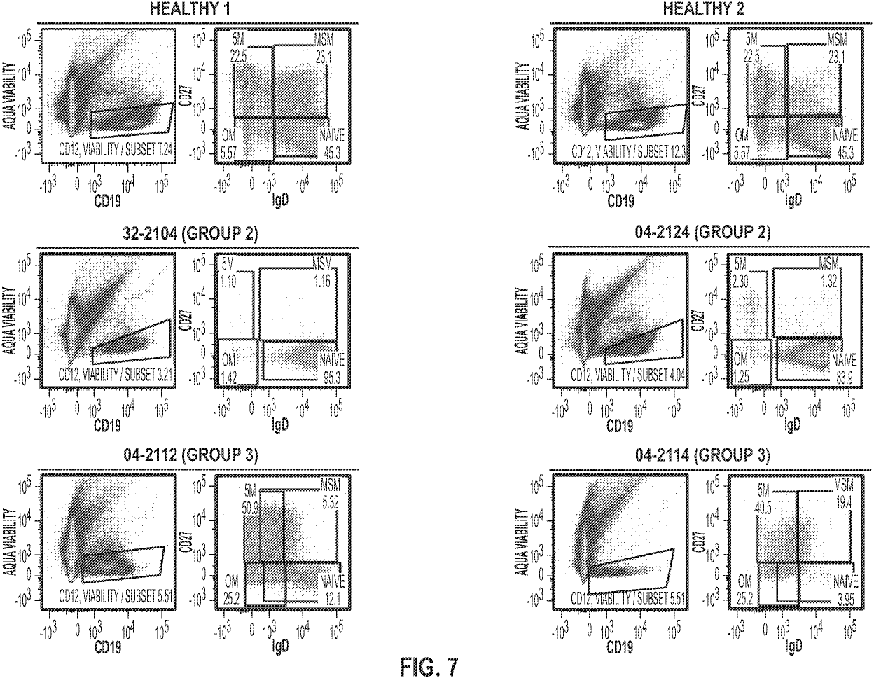

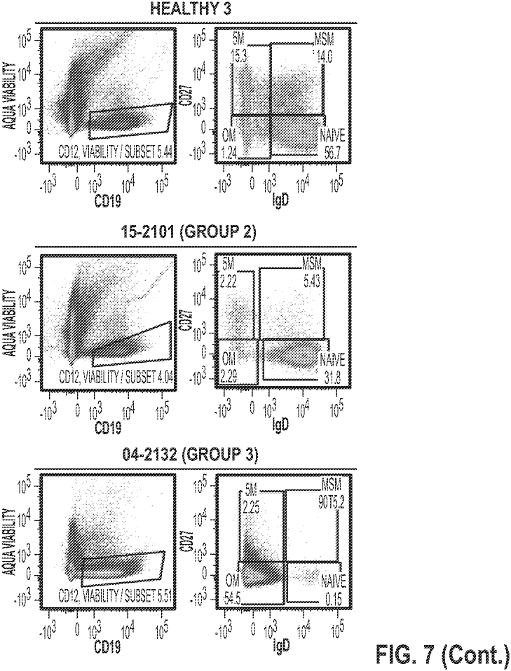

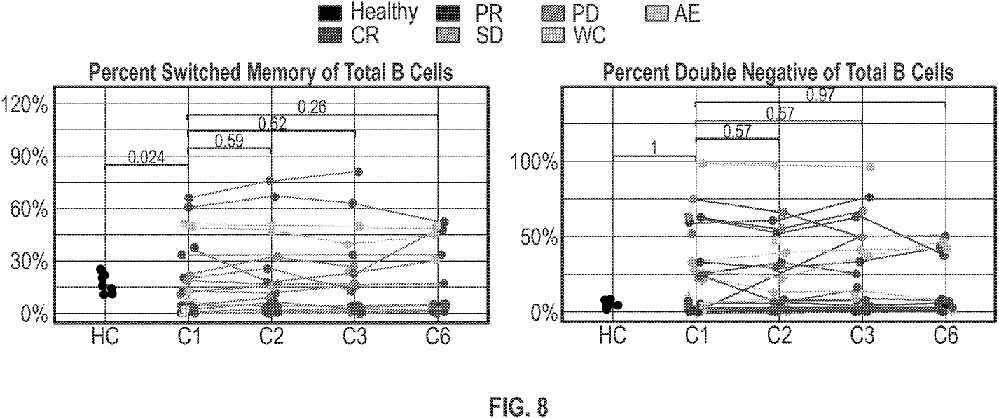

The cell population frequencies that drive the separation between Group 3 and Groups 1 and 2 were evaluated. As shown in FIG. 6, while the percent B cells of lymphocytes (top left) is not substantively different between healthy and the three iNHL groups, both Groups 1 and 2 show clear elevations in naïve B cell population frequency while Group 3 presents with a dramatic loss in naïve B cell frequency of total B cells (top right). Also observed was a significant decrease in Groups 1 and 2 switched memory B cell frequency of total B cells, whereas Group 3 patients were not significantly different from healthy albeit with several patients demonstrating clear elevations (bottom left). Significant elevations in the double negative B cell frequency of total B cells was also uniquely observed in the Group 3 patients (bottom right). Raw FACS plots showing examples of this phenomenon are shown in FIG. 7. The top row represents 3 healthy controls, where we observe representation of double negative, naïve, switched memory, and non-switched memory B cells. Compared to Group 2 (and Group 1, not shown here), significant skewing of the total B cells to naïve B cell phenotype is found (middle row). Group 3 patients present with either a skewing towards high frequency switched memory or double negative (bottom row). Shown in FIG. 8, as with other B cell subsets, that cerdulatinib had no meaningful impact on the frequencies of switched memory or double negative B cells over time.

The disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains. This description is not intended to limit the invention to the precise form disclosed. It is intended that the scope of the invention be defined by the claims appended hereto.

What is claimed is:

1. A method for treating a B cell lymphoma, comprising administering a kinase inhibitor to a patient suffering from the B cell lymphoma, wherein the kinase inhibitor is selected from the group consisting of a PI3K inhibitor, a SYK inhibitor, a JAK inhibitor, and a BTK inhibitor, and wherein the patient
(a) does not have increased frequency of transitional (CD10+) B cells within total naive B cells or within total B cells; or
(b) has decreased frequency of naive B cells within total B cells, and has increased frequency of memory switched B cells or double negative B cells within total B cells,
wherein the increase and decrease are as compared to a corresponding healthy subject not having the B cell lymphoma.

2. The method of claim 1, wherein no more than 30% of the naive B cells of the patient are transitional B cells.

3. The method of claim 1, wherein no more than 25% of the total B cells of the patient are transitional B cells.

4. The method of any preceding claim, wherein the B cell lymphoma is non-Hodgkin lymphoma (NHL) .

5. The method of claim 4, wherein the NHL is indolent NHL (iNHL).

6. The method of any one of claims 1-5, wherein the frequencies are determined in a blood sample obtained or derived from the patient.

7. The method of claim 6, further comprising:
obtaining or preparing the blood sample; and
determining, in the blood sample, numbers of total B cells, transitional B cells, naive B cells, double negative B cells, switched memory B cells, or any combination thereof.

8. The method of any preceding claim, wherein the transitional B cells comprises CD10+ transitional B cells, or CD38+ transitional B cells, or both.

9. A method for treating a B cell lymphoma, comprising:
determining, in a blood sample isolated from a B cell lymphoma patient, numbers of total B cells, transitional B cells and/or naive B cells;
selecting the patient for treatment when the patient (a) does not have increased frequency of transitional (CD10+) B cells within total naive B cells or within total B cells; or (b) has decreased frequency of naive B cells within total B cells, and has increased frequency of memory switched B cells or double negative B cells within total B cells, wherein the increase and decrease are as compared to a corresponding healthy subject not having the B cell lymphoma; and
administering a kinase inhibitor selected from the group consisting of a PI3K inhibitor, a SYK inhibitor, a JAK inhibitor, and a BTK inhibitor to the patient.

10. A method for identifying a patient as suitable for treatment with a kinase inhibitor, comprising
determining, in a blood sample derived from a B cell lymphoma patient, the numbers of total B cells, transitional B cells and/or naive B cells,
wherein the patient is identified as suitable for the treatment if the patient (a) does not have increased frequency of transitional (CD10+) B cells within total naive B cells or within total B cells; or (b) has decreased frequency of naive B cells within total B cells, and has increased frequency of memory switched B cells or double negative B cells within total B cells,
wherein the increase and decrease are as compared to a corresponding healthy subject not having the B cell lymphoma, and
wherein the kinase inhibitor is selected from the group consisting of a PI3K inhibitor, a SYK inhibitor, a JAK inhibitor, and a BTK inhibitor.

11. The method of claim 9 or 10, wherein the patient selected or identified has no more than 30% of the naive B cells being transitional B cells.

12. The method of claim 9 or 10, wherein the patient has no more than 25% of the total B cells being transitional B cells.

13. The method of any one of claims 9-12, wherein the B cell lymphoma is non-Hodgkin lymphoma (NHL) .

14. The method of claim 13, wherein the NHL is indolent NHL (iNHL).

15. The method of any preceding claim, wherein the patient has not suffered from a diabetic or pre-diabetic condition prior to the administration.

16. The method of any preceding claim, wherein the patient has had fewer than 5 prior treatments for the B cell lymphoma.

17. The method of any preceding claim, wherein the patient has not had any prior treatment for the B cell lymphoma.

18. The method of any preceding claim, wherein the kinase inhibitor is a PI3K inhibitor selected from the group consisting of idelalisib, IPI-145, AMG-319, TGR-1202, and BKM120.

19. The method of any one of claims 1-17, wherein the kinase inhibitor is a SYK inhibitor selected from the group consisting of fostamatinib, entospletinib, cerdulatinib, and TAK-659.

20. The method of any one of claims 1-17, wherein the kinase inhibitor is a JAK inhibitor selected from the group consisting of ruxolitinib, tofacitinib, oclacitinib, baricitinib, peficitinib, fedratinib, upadacitinib, and cerdulatinib.

21. The method of any one of claims 1-17, wherein the kinase inhibitor is a BTK inhibitor.

22. The method of any one of claims 1-17, wherein the kinase inhibitor is cerdulatinib.
